(19) United States
(12) Patent Application Publication
(10) Pub. No.: US 2026/0041757 A1
(43) Pub. Date: Feb. 12, 2026

(54) THERMOSTABLE MUCOSAL VACCINE COMPOSITIONS AND METHODS

(71) Applicant: GLOBAL HEALTH SOLUTIONS, INC., Westlake Village, CA (US)

(72) Inventor: Bradley Burnam, Calabasas, CA (US)

(21) Appl. No.: 19/295,239

(22) Filed: Aug. 8, 2025

Related U.S. Application Data
(60) Provisional application No. 63/681,528, filed on Aug. 9, 2024.

(51) Int. Cl. A61K 39/205; A61K 39/00; A61K 39/04; A61K 39/12; A61K 39/215

(52) U.S. Cl. CPC A61K 39/205; A61K 39/04; A61K 39/12; A61K 39/215; A61K 2039/542; A61K 2039/60; C12N 2760/10034; C12N 2760/14134; C12N 2760/14234; C12N 2760/20034; C12N 2770/20034

(57) ABSTRACT

Methods for stabilizing a liquid vaccine for mucosal use are provided. In certain aspects, the methods may include combining a liquid comprising at least one antigen or antigen encoding composition with a petrolatum carrier.

### CROSS-REFERENCE TO RELATED APPLICATIONS

This application claims the benefit of U.S. Provisional Application Ser. No. 63/681,528, entitled “Thermostable Mucosal Vaccine Compositions and Methods,” filed Aug. 9, 2024, the contents of which are incorporated by reference herein, for all purposes, in their entirety.

### TECHNICAL FIELD

The present disclosure generally relates to thermostable vaccine compositions and methods for the thermostable delivery of mucosal vaccines, particularly for intranasal or mucosal administration through oral, nasal, rectal, or vaginal immunization routes.

### BACKGROUND

The majority of infectious agents first contact and enter animal bodies through a mucosal surface. Accordingly, mucosal immune responses constitute a primary defense against infectious agents and offer the potential to effect strong protective immune response at the predominant sites of pathogen infection. In particular, mucosal immune responses may be induced by mucosal immunization through nasal, oral, rectal, or vaginal routes. Nonetheless, the vast majority of vaccines in use today are administered by intramuscular or subcutaneous injection despite the fact that such vaccines often create a poor mucosal immune response. A significant barrier to the adoption and use of mucosal vaccines arises from the difficulty of maintaining vaccine stability during administration and storage of mucosal vaccines. Most vaccines have poor thermostability and require uninterrupted cold storage at 2-8° C. from the time the vaccine is manufactured until the time it is administered to the patient. The need for cold storage of vaccines often accounts for the majority of the cost of vaccination programs worldwide and reduces their effective use in areas suffering from poor infrastructure.

Vaccine efficacy and potency requires proper maintenance of unbroken cold chain from vaccine manufacturing through vaccine distribution, vaccine arrival at a health provider facility, vaccine storage and handling at the provider facility, until vaccine administration. The result of a cold chain failure can be costly as well as damage public health due to the administration of vaccines that are either ineffective or have reduced effectiveness.

Additionally, even during relatively short storage periods in cold storage at 2-8° C., vaccines may be damaged or experience reduced potency due to the occurrence of one or more non-ideal storage scenarios or as a result of human error. For example, vaccines nominally stored in cold storage at 2-8° C. may nonetheless experience temperature excursions outside of the 2-8° C. temperature range due to too frequent opening and closing of the refrigeration unit door, incorrect positioning of the vaccines within the refrigeration unit, incorrect or deficient use of thermal mass (e.g., water bottles) to mitigate against door opening and closings, thermostat failure, poor temperature monitoring, electrical power loss or interruption, improper setting of the thermostat temperature at a point other than the midpoint, improper calibration, alarm failures, high ambient temperatures outside the refrigeration unit, and the use of refrigeration units not purpose built for the storage of biologics. Even relatively small temperature excursions can damage or reduce the potency of vaccines. For example, accidental freezing of the vaccine during cold storage can damage or reduce the effectiveness of some vaccines. Additionally, the vaccination of populations away from clinical environments and in resource-limiting settings often necessitate storing vaccines at ambient conditions for a significant period of time prior to administration.

Compared with other vaccines, mRNA vaccines are particularly unstable stemming from the inherent fragility of the mRNA molecule as well as its interaction with and reliance on lipid nanoparticles in the formulation. Accordingly, mRNA vaccines must be carefully transported and stored prior to administration resulting and are especially susceptible to temperature excursions and nonideal administration conditions.

Therefore, the Applicant has recognized the need for the development of vaccine compositions and methods, particularly mucosal vaccine compositions and methods, having increased thermostability in order to increase vaccine storage duration in cold storage and/or ambient environments prior to administration, greatly reduce vaccination costs, reduce damage or potency loss due to non-ideal cold storage, improve accessibility, and reduce the barrier to mucosal vaccine adoption. Additionally, the administration of mucosal vaccines often suffers from excessive transfer losses as well as a resultant reduction in dosing predictability. Accordingly, improved mucosal vaccine compositions that are characterized by improved stability at ambient temperatures and/or improved vaccine transfer or dosing predictability at the mucosal administration site are desirable.

### SUMMARY

To address these shortcomings, Applicant has discovered new compositions and methods for providing thermostable mucosal vaccine storage and administration. The presently disclosed compositions and methods have surprisingly been discovered to increase the stability, particularly the thermal stability, of liquid mucosal vaccines thereby extending the time period by which the vaccines may be stored in cold storage or at ambient conditions prior to administration to a subject, without an accompanying loss in vaccine effectiveness or potency. Additionally, the presently disclosed compositions and methods are effective in providing thermal protection to liquid mucosal vaccines against non-ideal storage conditions and unwanted temperature excursions during cold storage, including protection against accidental freezing. The Applicant has surprisingly discovered that petrolatum carrier may act as an insulator reducing or delaying heat uptake, while increasing thermal mass and resistance to temperature excursions, and helping to prevent unwanted freezing that may result from temperature excursions during cold storage. Additionally, the Applicant has also unexpectedly discovered that the presently disclosed methods and compositions may further provide for mucosal vaccine delivery with substantially less transfer loss and with greater dosing predictability, as a result of increased contact duration of time over which the vaccine may be taken up by the mucosal site. Among other embodiments, the presently disclosed compositions and methods provide a novel method of suspending live viral vectors, including both lipid-enveloped and non-lipid-enveloped viruses, in a petrolatum-based carrier along with various buffer solutions. This approach enhances the thermostability of vaccine compositions and enables them to remain viable for extended periods of time under refrigerated conditions and for additional durations at room temperature or ambient conditions. In certain embodiments, the presently disclosed compositions and methods may eliminate the need for freezers and facilitate easier transport and deployment of vaccines in challenging environments, such as in vehicles or remote field stations. This method may be particularly advantageous for intranasal delivery, making it suitable for a range of viruses and vaccines, including but not limited to VSV vectors (e.g., Ebola, Marburg), COVID-19, and other respiratory diseases. Additionally, it could be applied to protein-based vaccines, such as pneumococcal and polio, expanding applicability across multiple disease targets and improving vaccine delivery in resource-limited settings.

The present disclosure provides methods of stabilizing a liquid vaccine for mucosal use. In particular, the present disclosure provides methods of increasing the stability of a liquid vaccine for mucosal use during storage or prior to the administration of the vaccine to a subject in need of vaccination. The presently disclosed methods also provide for increasing the resistance to potency loss of a liquid vaccine for mucosal use during storage or prior to administration to a subject. These methods may include combining a liquid comprising at least one antigen or antigen encoding composition with a petrolatum carrier.

The present disclosure also provides methods for increasing the time period that a liquid vaccine for mucosal use may be stored prior to administration to a subject in need thereof without a substantial loss in potency or effectiveness. Such methods include combining a liquid comprising at least one antigen or antigen encoding composition with a petrolatum carrier. In particular, the combining of the liquid comprising at least one antigen or antigen encoding composition with the petrolatum carrier may form a stabilized mucosal vaccine. According to certain aspects of the present disclosure, the method may further include storing the stabilized mucosal vaccine at a temperature of from about 2 degrees Celsius to about 8 degrees Celsius for a predetermined period of time prior to administration to the subject. In certain instances, the predetermined period of time may be greater than 30 days. In other instances, the predetermined period of time may be greater than 60 days, or greater than 90 days, or greater than 120 days, or at least 3 months. In other embodiments in which particularly temperature sensitive vaccines are employed, the predetermined period of time may be greater than 6 hours, or greater than 8 hours, or greater than 10 hours, or greater than 12 hours, or greater than 24 hours.

The present disclosure also provides methods for increasing the stability of a liquid vaccine for mucosal use during temperature excursions associated with cold storage of the vaccine prior to administration of the vaccine to a subject. The present disclosure also provides methods for increasing the resistance to potency loss of a liquid vaccine for mucosal use during temperature excursions associated with cold storage of the vaccine. In such methods, the method may include storing a stabilized mucosal vaccine that has been prepared by combining a liquid comprising at least one antigen or antigen encoding composition with a petrolatum carrier at a temperature of from about 2 degrees Celsius to about 8 degrees Celsius for a predetermined period of time prior to administration to the subject. In certain instances, the methods may also include combining a liquid comprising at least one antigen or antigen encoding composition with a petrolatum carrier to form the stabilized mucosal vaccine. In certain instances, the predetermined period of time may be greater than 30 days. In other instances, the predetermined period of time may be greater than 60 days, or greater than 90 days, or greater than 120 days, or at least 3 months. In other embodiments in which particularly temperature sensitive vaccines are employed, the predetermined period of time may be greater than 6 hours, or greater than 8 hours, or greater than 10 hours, or greater than 12 hours, or greater than 24 hours.

The present disclosure also provides methods for increasing the time period that a liquid vaccine for mucosal use may be stored in a cold storage refrigeration unit prior to administration to a subject in need thereof without a substantial loss in potency or effectiveness. For example, the methods may include storing a stabilized mucosal vaccine that has been prepared by combining a liquid comprising at least one antigen or antigen encoding composition with a petrolatum carrier in a cold storage refrigeration unit for a predetermined amount of time prior to administration to the subject. The cold storage refrigeration unit may be designed or set to maintain a temperature within the unit of from 2 degrees Celsius to 8 degrees Celsius. In certain instances, the predetermined period of time may be greater than 30 days. In other instances, the predetermined period of time may be greater than 60 days, or greater than 90 days, or greater than 120 days, or at least 3 months. In other embodiments in which particularly temperature sensitive vaccines are employed, the predetermined period of time may be greater than 6 hours, or greater than 8 hours, or greater than 10 hours, or greater than 12 hours, or greater than 24 hours.

The present disclosure also provides methods for vaccinating a subject or otherwise causing an immune response in a subject. These methods may include administering a stabilized mucosal vaccine that was prepared by combining a liquid comprising at least one antigen or antigen encoding composition with a petrolatum carrier to the mucosa or a mucosal membrane of the subject. In some instances, these methods may further include combining a liquid comprising at least one antigen or antigen encoding composition with a petrolatum carrier to form the stabilized mucosal vaccine. Administering the stabilized mucosal vaccine to the mucosa or a mucosal membrane of the subject may include oral, nasal, rectal, or vaginal administration. In at least some instances, administering the stabilized mucosal vaccine may include intranasal administration of the stabilized mucosal vaccine.

The present disclosure also provides methods for increasing the dosing predictability of a mucosal vaccine as well as methods for increasing the dose transfer of a mucosal vaccine or vaccine administered via a mucosal administration route. These methods may include administering a stabilized mucosal vaccine that was prepared by combining a liquid comprising at least one antigen or antigen encoding composition that has with a petrolatum carrier to the mucosa or a mucosal membrane of the subject. In some instances, these methods may further include combining a liquid comprising at least one antigen or antigen encoding composition with a petrolatum carrier to form the stabilized mucosal vaccine. Administering the stabilized mucosal vaccine to the mucosa or a mucosal membrane of the subject may include oral, nasal, rectal, or vaginal administration. In at least some instances, administering the stabilized mucosal vaccine may include intranasal administration of the stabilized mucosal vaccine.

The present disclosure also provides methods for preparing a mucosal vaccine with increased dosing predictability as well as methods for preparing a mucosal vaccine with increased dose transfer. These methods may include combining a liquid comprising at least one antigen or antigen encoding composition with a petrolatum carrier to form the mucosal vaccine operable to exhibit increased dosing predictability and/or increased dose transfer once administered to a subject via a mucosal administration route.

Still other aspects and advantages of these exemplary embodiments and other embodiments, are discussed in detail herein. Moreover, it is to be understood that both the foregoing information and the following detailed description provide merely illustrative examples of various aspects and embodiments and are intended to provide an overview or framework for understanding the nature and character of the claimed aspects and embodiments. Accordingly, these and other advantages and features of the present disclosure, will become apparent through reference to the following description and the accompanying drawings. Furthermore, it is to be understood that the features of the various embodiments described herein are not mutually exclusive and may exist in various combinations and permutations.

### DETAILED DESCRIPTION

The present disclosure describes various embodiments related to methods and compositions for stabilizing a liquid vaccine for mucosal use, in particular, for increasing the time period that a liquid vaccine for mucosal use may be stored prior to administration to a subject as well as increasing the resistance to potency loss of a liquid vaccine for mucosal use during storage or as a result of unwanted temperature excursions during storage. Further embodiments may be described and disclosed.

In the following description, numerous details are set forth in order to provide a thorough understanding of the various embodiments. In other instances, well-known processes, devices, and systems may not have been described in particular detail in order not to unnecessarily obscure the various embodiments. Additionally, illustrations of the various embodiments may omit certain features or details in order to not obscure the various embodiments.

The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “reducing,” “reduced,” or any variation thereof, when used in the claims and/or the specification includes any measurable decrease or complete inhibition to achieve a desired result.

The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

The present disclosure provides methods of stabilizing vaccine compositions for mucosal use. The vaccine compositions are generally liquid vaccines for mucosal use. In particular, the present disclosure provides methods of increasing the stability of a liquid vaccine for mucosal use during storage or prior to the administration of the vaccine to a subject in need of vaccination. The presently disclosed methods also provide for increasing the resistance to potency loss of a liquid vaccine for mucosal use during storage or prior to administration to a subject. These methods may include combining a liquid comprising at least one antigen or antigen encoding composition with a petrolatum carrier. Non-limiting examples of antigen encoding compositions include compositions that include mRNA or other molecules that when introduced into a subject instructs cells in the subject to produce a specific antigen or protein which in turn triggers an immune response in the subject. According to certain embodiments of the present disclosure, antigen encoding compositions include mRNA vaccines and compositions thereof.

The present disclosure also provides methods for increasing the time period that a liquid vaccine for mucosal use may be stored prior to administration to a subject in need thereof without a substantial loss in potency or effectiveness. Such methods include combining a liquid comprising at least one antigen or antigen encoding composition with a petrolatum carrier. In particular, the combining of the liquid comprising at least one antigen or antigen encoding composition with the petrolatum carrier may form a stabilized mucosal vaccine. According to certain aspects of the present disclosure, the method may further include storing the stabilized mucosal vaccine at a temperature of from about 2 degrees Celsius to about 8 degrees Celsius for a predetermined period of time prior to administration to the subject. In certain instances, the predetermined period of time may be greater than 30 days. In other instances, the predetermined period of time may be greater than 60 days, or greater than 90 days, or greater than 120 days, or at least 3 months. In other embodiments in which particularly temperature sensitive vaccines are employed, the predetermined period of time may be greater than 6 hours, or greater than 8 hours, or greater than 10 hours, or greater than 12 hours, or greater than 24 hours.

According to other aspects of the present disclosure, the methods may include storing the stabilized mucosal vaccine at a temperature of from about 8.5 degrees Celsius to about 25 degrees Celsius for a predetermined period of time prior to administration to the subject. In certain instances, the predetermined period of time may be greater than 2 hours. In other instances, the predetermined period of time may be greater than 6 hours, or greater than 8 hours, or greater than 10 hours, or greater than 12 hours.

According to other aspects of the present disclosure, the methods may include storing the stabilized mucosal vaccine at a temperature of from about 20 degrees Celsius to about 25 degrees Celsius for a predetermined period of time prior to administration to the subject. In certain instances, the predetermined period of time may be greater than 2 hours. In other instances, the predetermined period of time may be greater than 6 hours, or greater than 8 hours, or greater than 10 hours, or greater than 12 hours.

The present disclosure also provides methods for increasing the stability of a liquid vaccine for mucosal use during temperature excursions associated with cold storage of the vaccine prior to administration of the vaccine to a subject. The present disclosure also provides methods for increasing the resistance to potency loss of a liquid vaccine for mucosal use during temperature excursions associated with cold storage of the vaccine. In such methods, the method may include storing a stabilized mucosal vaccine that has been prepared by combining a liquid comprising at least one antigen or antigen encoding composition with a petrolatum carrier at a temperature of from about 2 degrees Celsius to about 8 degrees Celsius for a predetermined period of time prior to administration to the subject. In certain instances, the methods may also include combining a liquid comprising at least one antigen or antigen encoding composition with a petrolatum carrier to form the stabilized mucosal vaccine. In certain instances, the predetermined period of time may be greater than 30 days. In other instances, the predetermined period of time may be greater than 60 days, or greater than 90 days, or greater than 120 days, or at least 3 months. In other embodiments in which particularly temperature sensitive vaccines are employed, the predetermined period of time may be greater than 6 hours, or greater than 8 hours, or greater than 10 hours, or greater than 12 hours, or greater than 24 hours.

The present disclosure also provides methods for increasing the time period that a liquid vaccine for mucosal use may be stored in a cold storage refrigeration unit prior to administration to a subject in need thereof without a substantial loss in potency or effectiveness. For example, the methods may include storing a stabilized mucosal vaccine that has been prepared by combining a liquid comprising at least one antigen or antigen encoding composition with a petrolatum carrier in a cold storage refrigeration unit for a predetermined amount of time prior to administration to the subject. In certain instances, the methods may also include combining a liquid comprising at least one antigen or antigen encoding composition with a petrolatum carrier to form the stabilized mucosal vaccine. The cold storage refrigeration unit may be designed or set to maintain a temperature within the unit of from 2 degrees Celsius to 8 degrees Celsius. In certain instances, the predetermined period of time may be greater than 30 days. In other instances, the predetermined period of time may be greater than 60 days, or greater than 90 days, or greater than 120 days, or at least 3 months. In other embodiments in which particularly temperature sensitive vaccines are employed, the predetermined period of time may be greater than 6 hours, or greater than 8 hours, or greater than 10 hours, or greater than 12 hours, or greater than 24 hours.

The present disclosure also provides methods for increasing the resistance to potency loss of a liquid vaccine for mucosal use during temporary storage of the vaccine at room temperature prior to administration of the vaccine to a subject. The present disclosure also provides methods for increasing the time period that a liquid vaccine for mucosal use may be temporarily stored at room temperature prior to administration to a subject in need thereof without a substantial loss in potency or effectiveness. In such methods, the method may include storing a stabilized mucosal vaccine that has been prepared by combining a liquid comprising at least one antigen or antigen encoding composition with a petrolatum carrier at a temperature of from about 20 degrees Celsius to about 25 degrees Celsius for a predetermined period of time prior to administration to the subject. In certain instances, the methods may also include combining a liquid comprising at least one antigen or antigen encoding composition with a petrolatum carrier to form the stabilized mucosal vaccine. In certain instances, the predetermined period of time may be greater than 2 hours. In other instances, the predetermined period of time may be greater than 6 hours, or greater than 8 hours, or greater than 10 hours, or greater than 12 hours.

The present disclosure also provides methods for vaccinating a subject or otherwise causing an immune response in a subject. These methods may include administering a stabilized mucosal vaccine that was prepared by combining a liquid comprising at least one antigen or antigen encoding composition that has with a petrolatum carrier to the mucosa or a mucosal membrane of the subject. In some instances, these methods may further include combining a liquid comprising at least one antigen or antigen encoding composition with a petrolatum carrier to form the stabilized mucosal vaccine. Administering the stabilized mucosal vaccine to the mucosa or a mucosal membrane of the subject may include oral, nasal, rectal, or vaginal administration. In at least some instances, administering the stabilized mucosal vaccine may include intranasal administration of the stabilized mucosal vaccine.

The present disclosure also provides methods for increasing the dosing predictability of a muscosal vaccine as well as methods for increasing the dose transfer of a mucosal vaccine or vaccine administered via a mucosal administration route. These methods may include administering a stabilized mucosal vaccine that was prepared by combining a liquid comprising at least one antigen or antigen encoding composition that has with a petrolatum carrier to the mucosa or a mucosal membrane of the subject. In some instances, these methods may further include combining a liquid comprising at least one antigen or antigen encoding composition with a petrolatum carrier to form the stabilized mucosal vaccine. Administering the stabilized mucosal vaccine to the mucosa or a mucosal membrane of the subject may include oral, nasal, rectal, or vaginal administration. In at least some instances, administering the stabilized mucosal vaccine may include intranasal administration of the stabilized mucosal vaccine.

The present disclosure also provides methods for preparing a mucosal vaccine with increased dosing predictability as well as methods for preparing a muscosal vaccine with increased dose transfer. These methods may include combining a liquid comprising at least one antigen or antigen encoding composition with a petrolatum carrier to form the mucosal vaccine operable to exhibit increased dosing predictability and/or increased dose transfer once administered to a subject via a mucosal administration route.

In the presently disclosed compositions and methods, the liquid comprising at least one antigen or antigen encoding composition may be any liquid vaccine suitable for mucosal administration. The at least one antigen or antigen encoding composition may be selected from the group consisting of a live attenuated vaccine (LAV), an inactivated (killed antigen) vaccine, a subunit (purified antigen) vaccine, a toxoid (inactivated toxin) vaccine, a bacteriophage, an antigen carried or delivered by a bacteriophage, an antigen carried or delivered by a recombinant vesicular stomatitis virus (rVSV) vector, and any combination thereof. In at least some instances, the at least one antigen or antigen encoding composition may be operable, upon administration to a subject, to cause an immune response in a subject sufficient to prevent, mitigate, or treat one or more infections selected from the group consisting of Marburg Virus, Ebola Zaire, Ebola Sudan, Lassa Virus, Buruli Ulcers, Leprosy, SARS-CoV-2 virus, and any combination thereof. For example, the liquid comprising at least one antigen or antigen encoding composition may be a liquid vaccine effective for the prevention, mitigation, or treatment of Marburg Virus, Ebola Zaire, Ebola Sudan, Lassa Fever, Buruli Ulcers, Leprosy, SARS-CoV-2 virus, and any combination thereof.

The presently disclosed stabilized mucosal vaccine compositions may include from about 10% by weight to about 90% by weight petrolatum carrier, or from about 5% by weight to about 95% by weight petrolatum carrier, or from about 10% by weight to about 95% by weight petrolatum carrier, or from about 35% by weight to about 95% by weight petrolatum carrier, or from about 35% by weight to about 90% by weight petrolatum carrier, or from about 10% by weight to about 35% by weight petrolatum carrier, from about 35% by weight to about 60% by weight petrolatum carrier, or from about 40% by weight to about 60% by weight petrolatum carrier, or from about 40% by weight to about 90% by weight petrolatum carrier, or at least 80% by weight petrolatum carrier, or at least 85% by weight petrolatum carrier, or at least 90% by weight petrolatum carrier, or up to 95% by weight petrolatum carrier.

The liquid comprising at least one antigen or antigen encoding composition may be prepared by dissolving or combining at least one antigen or antigen encoding composition in a liquid to provide a liquid comprising at least one antigen or antigen encoding composition. The liquid comprising at least one antigen or antigen encoding composition may also include one or more additional components commonly found in vaccine compositions. For example, the liquid comprising at least one antigen or antigen encoding composition may include preservatives, adjuvants, stabilizers, cell culture materials, inactivating ingredients, and/or antibiotics. Non-limiting examples of cell culture materials or culture media that may be present in the liquid include fetal bovine serum (FBS), Dulbecco's m Modified Eagle Medium (DMEM), Medium 199, and Minimum Essential Medium. The liquid comprising at least one antigen or antigen encoding composition may also include one or more adjuvants, such as aluminum salts, that help stimulate a stronger immune response to the vaccine antigens. The liquid may also include one or more stabilizers, such as sugars or gelatin, to help maintain the vaccine's potency during storage and transportation. For example, the liquid may include one or more components selected from the group consisting of lactose, sucrose, glycine, monosodium glutamate, human serum albumin, and bovine serum albumin. The liquid may also include one or more polar solvents, such as water. The liquid comprising at least one antigen or antigen encoding composition further may further include one or more preservatives. For example, the liquid may include sodium chloride or one or more cationic biocides. The one or more cationic biocides is selected from the group consisting of benzalkonium chloride, cetrimide, chlorhexidine, polihexanide biguanide, and any combination thereof.

The combining a liquid comprising at least one antigen or antigen encoding composition with a petrolatum carrier may result in the liquid being dispersed or suspended in the petrolatum carrier. In at least some instances, the liquid comprising at least one antigen or antigen encoding composition with a petrolatum carrier may be combined by one or more methods capable of causing the liquid to be suspended in the petrolatum carrier. In some instances, the liquid comprising at least one antigen or antigen encoding composition may be suspended or substantially suspended in the petrolatum carrier in the absence of an emulsifier or without the use of an added emulsifier. In other instances, the liquid comprising at least one antigen or antigen encoding composition may be dispersed or substantially dispersed in the petrolatum carrier in the absence of an emulsifier or without the use of an added emulsifier. For example, the liquid comprising at least one antigen or antigen encoding composition may be dispersed or substantially dispersed in the petrolatum carrier in the form of nanodroplets dispersed or suspended in the petrolatum carrier.

The presently disclosed mucosal vaccine compositions may be prepared by: a) providing a liquid comprising at least one antigen or antigen encoding composition; b) heating petrolatum or a composition comprising petrolatum to a temperature sufficient to cause the petrolatum to melt to give a melted petrolatum carrier composition; c) mixing the melted petrolatum carrier composition and the liquid comprising at least one antigen or antigen encoding composition to give a melted mixture; and d) cooling the melted mixture to provide a petrolatum mucosal vaccine composition.

In other instances, the presently disclosed mucosal vaccine compositions may be prepared by: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) mixing the melted petrolatum carrier composition and the liquid comprising at least one antigen or antigen encoding composition to give a melted mixture; and d) allowing the melted mixture to cool to room temperature to provide a petrolatum mucosal vaccine composition.

In still other instances, the presently disclosed mucosal vaccine compositions may be prepared by: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) heating the liquid comprising at least one antigen or antigen encoding composition to a temperature equal to or less than the temperature of the melted petrolatum carrier composition to give a heated liquid antigen composition; d) mixing the melted petrolatum carrier composition and the heated liquid antigen composition to give a melted mixture; and e) allowing the melted mixture to cool to room temperature to provide a petrolatum mucosal vaccine composition.

In yet other instances, the presently disclosed mucosal vaccine compositions may be prepared by: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) heating the liquid comprising at least one antigen or antigen encoding composition to a temperature that is about 1 degree C. to about 5 degrees Celsius higher than the temperature of the melted petrolatum carrier composition to give a heated liquid antigen composition; d) mixing the melted petrolatum carrier composition and the heated liquid antigen composition to give a melted mixture; and e) allowing the melted mixture to cool to room temperature to provide a petrolatum mucosal vaccine composition.

### EXAMPLES

The examples provided below illustrates selected aspects of the presently disclosed methods.

### Example 1—Method of Stabilizing a Liquid Vaccine for Mucosal Use for Extended Storage at 2° C.-8° C. Prior to Administration to a Subject

A liquid mucosal vaccine comprising at least one antigen or antigen encoding composition will be mixed with sufficient petrolatum carrier so as to confer additional thermostability to the resultant thermostable mucosal vaccine composition so that that the vaccine will remain stable and effective for achieving the desired immune response in a subject following at least 30 days storage in a refrigeration unit set to maintain a temperature from 2 degrees Celsius to 8 degrees Celsius.

### Example 2—Method of Stabilizing a Liquid Vaccine for Mucosal Use for Extended Storage at 2° C.-8° C. Prior to Administration to a Subject

A liquid mucosal vaccine comprising at least one antigen or antigen encoding composition will be mixed with sufficient petrolatum carrier so as to confer additional thermostability to the resultant thermostable mucosal vaccine composition so that that the vaccine will remain stable and effective for achieving the desired immune response in a subject following at least 24 hours storage in a refrigeration unit set to maintain a temperature from 2 degrees Celsius to 8 degrees Celsius.

### Example 3—Method of Stabilizing a Liquid Vaccine for Mucosal Use for Extended Storage at 8.5° C.-25° C. Prior to Administration to a Subject

A liquid mucosal vaccine comprising at least one antigen or antigen encoding composition will be mixed with sufficient petrolatum carrier so as to confer additional thermostability to the resultant thermostable mucosal vaccine composition so that that the vaccine will remain stable and effective for achieving the desired immune response in a subject following at least 6 hours storage in an ambient environment characterized by having a temperature of 8.5 degrees Celsius to 25 degrees Celsius.

### Example 4—Method of Stabilizing a Liquid Vaccine for Mucosal Use for Extended Storage at 20° C.-25° C. Prior to Administration to a Subject

A liquid mucosal vaccine comprising at least one antigen or antigen encoding composition will be mixed with sufficient petrolatum carrier so as to confer additional thermostability to the resultant thermostable mucosal vaccine composition so that that the vaccine will remain stable and effective for achieving the desired immune response in a subject following at least 2 hours storage in an ambient environment characterized by having a temperature of 20 degrees Celsius to 25 degrees Celsius.

### Example 5—Method of Stabilizing a Liquid Vaccine for Mucosal Use for Extended Storage at 2° C.-8° C. Prior to Administration to a Subject

A liquid mucosal vaccine comprising at least one antigen or antigen encoding composition will be mixed with petrolatum carrier such that the resultant thermostable mucosal vaccine composition comprises from about 10 weight percent to about 35 weight percent petrolatum carrier. The thermostable mucosal vaccine composition will remain stable and effective for achieving the desired immune response in a subject following at least 30 days storage in a refrigeration unit set to maintain a temperature from 2 degrees Celsius to 8 degrees Celsius.

### Example 6—Method of Stabilizing a Liquid Vaccine for Mucosal Use for Extended Storage at 2° C.-8° C. Prior to Administration to a Subject

A liquid mucosal vaccine comprising at least one antigen or antigen encoding composition will be mixed with petrolatum carrier such that the resultant thermostable mucosal vaccine composition comprises from about 35 weight percent to about 60 weight percent petrolatum carrier. The thermostable mucosal vaccine composition will remain stable and effective for achieving the desired immune response in a subject following at least 30 days storage in a refrigeration unit set to maintain a temperature from 2 degrees Celsius to 8 degrees Celsius.

### Example 7—Method of Stabilizing a Liquid Vaccine for Mucosal Use for Extended Storage at 2° C.-8° C. Prior to Administration to a Subject

A liquid mucosal vaccine comprising at least one antigen or antigen encoding composition will be mixed with petrolatum carrier such that the resultant thermostable mucosal vaccine composition comprises from about 35 weight percent to about 95 weight percent petrolatum carrier. The thermostable mucosal vaccine composition will remain stable and effective for achieving the desired immune response in a subject following at least 30 days storage in a refrigeration unit set to maintain a temperature from 2 degrees Celsius to 8 degrees Celsius.

### Example 8—Method of Stabilizing a Liquid Vaccine for Mucosal Use for Extended Storage at 2° C.-8° C. Prior to Administration to a Subject

A liquid mucosal vaccine comprising at least one antigen or antigen encoding composition will be mixed with petrolatum carrier such that the resultant thermostable mucosal vaccine composition comprises from about 35 weight percent to about 95 weight percent petrolatum carrier and the liquid comprising at least one antigen or antigen encoding composition is dispersed or suspended in the petrolatum carrier. The thermostable mucosal vaccine composition will remain stable and effective for achieving the desired immune response in a subject following at least 30 days storage in a refrigeration unit set to maintain a temperature from 2 degrees Celsius to 8 degrees Celsius.

### Example 9—Method of Preparing a Thermostable Mucosal Vaccine

A liquid mucosal vaccine comprising at least one antigen or antigen encoding composition will be mixed with petrolatum carrier such that the resultant thermostable mucosal vaccine composition comprises from about 35 weight percent to about 95 weight percent petrolatum carrier and the liquid comprising at least one antigen or antigen encoding composition is dispersed or suspended in the petrolatum carrier. The thermostable mucosal vaccine composition will be prepared by the following steps: a) providing a liquid comprising at least one antigen or antigen encoding composition; b) heating petrolatum or a composition comprising petrolatum to a temperature sufficient to cause the petrolatum to melt to give a melted petrolatum carrier composition; c) mixing the melted petrolatum carrier composition and the liquid comprising at least one antigen or antigen encoding composition to give a melted mixture; and d) cooling the melted mixture to provide a petrolatum mucosal vaccine composition.

The thermostable mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 10—Method of Preparing a Thermostable Mucosal Vaccine

A liquid mucosal vaccine comprising at least one antigen or antigen encoding composition will be mixed with petrolatum carrier such that the resultant thermostable mucosal vaccine composition comprises from about 35 weight percent to about 95 weight percent petrolatum carrier and the liquid comprising at least one antigen or antigen encoding composition is dispersed or suspended in the petrolatum carrier. The thermostable mucosal vaccine composition will be prepared by the following steps: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) mixing the melted petrolatum carrier composition and the liquid comprising at least one antigen or antigen encoding composition to give a melted mixture; and d) allowing the melted mixture to cool to room temperature to provide a petrolatum mucosal vaccine composition.

The thermostable mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 11—Method of Preparing a Thermostable Mucosal Vaccine

A liquid mucosal vaccine comprising at least one antigen or antigen encoding composition will be mixed with petrolatum carrier such that the resultant thermostable mucosal vaccine composition comprises from about 35 weight percent to about 95 weight percent petrolatum carrier and the liquid comprising at least one antigen or antigen encoding composition is dispersed or suspended in the petrolatum carrier. The thermostable mucosal vaccine composition will be prepared by the following steps: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) heating the liquid comprising at least one antigen or antigen encoding composition to a temperature equal to or less than the temperature of the melted petrolatum carrier composition to give a heated liquid antigen composition; d) mixing the melted petrolatum carrier composition and the heated liquid antigen composition to give a melted mixture; and d) allowing the melted mixture to cool to room temperature to provide a petrolatum mucosal vaccine composition.

The thermostable mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 12—Method of Preparing a Thermostable Mucosal Vaccine

A liquid mucosal vaccine comprising at least one antigen or antigen encoding composition will be mixed with petrolatum carrier such that the resultant thermostable mucosal vaccine composition comprises from about 35 weight percent to about 95 weight percent petrolatum carrier and the liquid comprising at least one antigen or antigen encoding composition is dispersed or suspended in the petrolatum carrier. The thermostable mucosal vaccine composition will be prepared by the following steps: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) heating the liquid comprising at least one antigen or antigen encoding composition to a temperature that is about 1 degree Celsius to about 5 degrees Celsius higher than the temperature of the melted petrolatum carrier composition to give a heated liquid antigen composition; d) mixing the melted petrolatum carrier composition and the heated liquid antigen composition to give a melted mixture; and d) allowing the melted mixture to cool to room temperature to provide a petrolatum mucosal vaccine composition.

The thermostable mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 13—Method of Preparing a Thermostable Marburg Virus Disease (MVC) Vaccine for Mucosal Administration

A liquid Marburg Virus Disease (MVD) vaccine formulated for mucosal administration will be mixed with petrolatum carrier such that the resultant thermostable Marburg Virus Disease (MVD) mucosal vaccine comprises from about 35 weight percent to about 95 weight percent petrolatum carrier and the liquid Marburg Virus Disease (MVD) vaccine is dispersed or suspended in the petrolatum carrier. The thermostable Marburg Virus Disease (MVD) mucosal vaccine composition will be prepared by the following steps: a) providing a liquid Marburg Virus Disease (MVD) vaccine; b) heating petrolatum or a composition comprising petrolatum to a temperature of from about 37 degrees Celsius to about 40 degrees Celsius to give a melted petrolatum carrier composition; c) heating the liquid vaccine to a temperature that is about 1 degree Celsius to about 5 degrees Celsius higher than the temperature of the melted petrolatum carrier composition to give a heated liquid antigen composition; d) mixing the melted petrolatum carrier composition and the heated liquid antigen composition to give a melted mixture; and d) allowing the melted mixture to cool to room temperature to provide a petrolatum mucosal vaccine composition.

The thermostable Marburg Virus Disease (MVD) mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 14—Method of Preparing a Thermostable Ebola Virus Vaccine for Mucosal Administration

A liquid Ebola virus vaccine (e.g., Ebola Zaire or Ebola Sudan) formulated for mucosal administration will be mixed with petrolatum carrier such that the resultant thermostable Ebola virus mucosal vaccine comprises from about 35 weight percent to about 95 weight percent petrolatum carrier and the liquid Ebola virus vaccine is dispersed or suspended in the petrolatum carrier. The thermostable Ebola virus mucosal vaccine composition will be prepared by the following steps: a) providing a liquid Ebola virus vaccine; b) heating petrolatum or a composition comprising petrolatum to a temperature of from about 37 degrees Celsius to about 40 degrees Celsius to give a melted petrolatum carrier composition; c) heating the liquid vaccine to a temperature that is about 1 degree Celsius to about 5 degrees Celsius higher than the temperature of the melted petrolatum carrier composition to give a heated liquid antigen composition; d) mixing the melted petrolatum carrier composition and the heated liquid antigen composition to give a melted mixture; and d) allowing the melted mixture to cool to room temperature to provide a petrolatum mucosal vaccine composition.

The thermostable Ebola virus mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 15—Method of Preparing a Thermostable Lassa Virus Vaccine for Mucosal Administration

A liquid Lassa virus vaccine formulated for mucosal administration will be mixed with petrolatum carrier such that the resultant thermostable Lassa virus mucosal vaccine comprises from about 35 weight percent to about 95 weight percent petrolatum carrier and the liquid Lassa virus vaccine is dispersed or suspended in the petrolatum carrier. The thermostable Lassa virus mucosal vaccine composition will be prepared by the following steps: a) providing a liquid Lassa virus vaccine; b) heating petrolatum or a composition comprising petrolatum to a temperature of from about 37 degrees Celsius to about 40 degrees Celsius to give a melted petrolatum carrier composition; c) heating the liquid vaccine to a temperature that is about 1 degree Celsius to about 5 degrees Celsius higher than the temperature of the melted petrolatum carrier composition to give a heated liquid antigen composition; d) mixing the melted petrolatum carrier composition and the heated liquid antigen composition to give a melted mixture; and d) allowing the melted mixture to cool to room temperature to provide a petrolatum mucosal vaccine composition.

The thermostable Lassa virus mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 16—Method of Preparing a Thermostable Buruli Ulcer Vaccine for Mucosal Administration

A liquid Buruli Ulcer vaccine formulated for mucosal administration will be mixed with petrolatum carrier such that the resultant thermostable Buruli Ulcer mucosal vaccine comprises from about 35 weight percent to about 95 weight percent petrolatum carrier and the liquid Buruli Ulcer vaccine is dispersed or suspended in the petrolatum carrier. The thermostable Buruli Ulcer mucosal vaccine composition will be prepared by the following steps: a) providing a liquid Buruli Ulcer vaccine; b) heating petrolatum or a composition comprising petrolatum to a temperature of from about 37 degrees Celsius to about 40 degrees Celsius to give a melted petrolatum carrier composition; c) heating the liquid vaccine to a temperature that is about 1 degree Celsius to about 5 degrees Celsius higher than the temperature of the melted petrolatum carrier composition to give a heated liquid antigen composition; d) mixing the melted petrolatum carrier composition and the heated liquid antigen composition to give a melted mixture; and d) allowing the melted mixture to cool to room temperature to provide a petrolatum mucosal vaccine composition.

The thermostable Buruli Ulcer mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 17—Method of Preparing a Thermostable Leprosy Vaccine for Mucosal Administration

A liquid Leprosy vaccine formulated for mucosal administration will be mixed with petrolatum carrier such that the resultant thermostable Leprosy mucosal vaccine comprises from about 35 weight percent to about 95 weight percent petrolatum carrier and the liquid Leprosy vaccine is dispersed or suspended in the petrolatum carrier. The thermostable Leprosy mucosal vaccine composition will be prepared by the following steps: a) providing a liquid Leprosy vaccine; b) heating petrolatum or a composition comprising petrolatum to a temperature of from about 37 degrees Celsius to about 40 degrees Celsius to give a melted petrolatum carrier composition; c) heating the liquid vaccine to a temperature that is about 1 degree Celsius to about 5 degrees Celsius higher than the temperature of the melted petrolatum carrier composition to give a heated liquid antigen composition; d) mixing the melted petrolatum carrier composition and the heated liquid antigen composition to give a melted mixture; and d) allowing the melted mixture to cool to room temperature to provide a petrolatum mucosal vaccine composition.

The thermostable Leprosy mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 18—Method of Preparing a Thermostable SARS-CoV-2 Vaccine for Mucosal Administration

A liquid SARS-CoV-2 vaccine formulated for mucosal administration will be mixed with petrolatum carrier such that the resultant thermostable SARS-CoV-2 mucosal vaccine comprises from about 35 weight percent to about 95 weight percent petrolatum carrier and the liquid SARS-CoV-2 vaccine is dispersed or suspended in the petrolatum carrier. The thermostable SARS-CoV-2 mucosal vaccine composition will be prepared by the following steps: a) providing a liquid SARS-CoV-2 vaccine; b) heating petrolatum or a composition comprising petrolatum to a temperature of from about 37 degrees Celsius to about 40 degrees Celsius to give a melted petrolatum carrier composition; c) heating the liquid vaccine to a temperature that is about 1 degree Celsius to about 5 degrees Celsius higher than the temperature of the melted petrolatum carrier composition to give a heated liquid antigen composition; d) mixing the melted petrolatum carrier composition and the heated liquid antigen composition to give a melted mixture; and d) allowing the melted mixture to cool to room temperature to provide a petrolatum mucosal vaccine composition.

The thermostable SARS-CoV-2 mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 19—Method of Preparing a Thermostable Vesicular Stomatitis Virus (rVSV) Vector Vaccine for Mucosal Administration

A liquid Vesicular Stomatitis Virus (rVSV) vector vaccine formulated for mucosal administration will be mixed with petrolatum carrier such that the resultant thermostable Vesicular Stomatitis Virus (rVSV) vector mucosal vaccine comprises from about 35 weight percent to about 95 weight percent petrolatum carrier and the liquid Vesicular Stomatitis Virus (rVSV) vector vaccine is dispersed or suspended in the petrolatum carrier. The thermostable Vesicular Stomatitis Virus (rVSV) vector mucosal vaccine composition will be prepared by the following steps: a) providing a liquid Vesicular Stomatitis Virus (rVSV) vector vaccine; b) heating petrolatum or a composition comprising petrolatum to a temperature of from about 37 degrees Celsius to about 40 degrees Celsius to give a melted petrolatum carrier composition; c) heating the liquid vaccine to a temperature that is about 1 degree Celsius to about 5 degrees Celsius higher than the temperature of the melted petrolatum carrier composition to give a heated liquid antigen composition; d) mixing the melted petrolatum carrier composition and the heated liquid antigen composition to give a melted mixture; and d) allowing the melted mixture to cool to room temperature to provide a petrolatum mucosal vaccine composition.

The thermostable Vesicular Stomatitis Virus (rVSV) vector mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 20—Method of Preparing a Thermostable Bacteriophage-Based Vaccine for Mucosal Administration

A liquid bacteriophage-based vaccine formulated for mucosal administration will be mixed with petrolatum carrier such that the resultant thermostable bacteriophage-based mucosal vaccine comprises from about 35 weight percent to about 95 weight percent petrolatum carrier and the liquid bacteriophage-based vaccine is dispersed or suspended in the petrolatum carrier. The thermostable bacteriophage-based mucosal vaccine composition will be prepared by the following steps: a) providing a liquid bacteriophage-based vector vaccine; b) heating petrolatum or a composition comprising petrolatum to a temperature of from about 37 degrees Celsius to about 40 degrees Celsius to give a melted petrolatum carrier composition; c) heating the liquid vaccine to a temperature that is about 1 degree Celsius to about 5 degrees Celsius higher than the temperature of the melted petrolatum carrier composition to give a heated liquid antigen composition; d) mixing the melted petrolatum carrier composition and the heated liquid antigen composition to give a melted mixture; and d) allowing the melted mixture to cool to room temperature to provide a petrolatum mucosal vaccine composition.

The thermostable bacteriophage-based vector mucosal vaccine composition will remain stable and effective for achieving the desired immune response in a subject following at least 30 days storage in a refrigeration unit set to maintain a temperature from 2 degrees Celsius to 8 degrees Celsius, and/or following at least 6 hours storage in an ambient environment characterized by having a temperature of 8.5 degrees Celsius to 25 degrees Celsius, and/or following at least 2 hours storage in an ambient environment characterized by having a temperature of 20 degrees Celsius to 25 degrees Celsius.

### Example 21—Preparation of Vaccine Composition

VSV-MARV-GP vaccine VPN1011 15% Trehalose in HBSS was combined with Sonneborn White Protopet 1S petrolatum according to the presently disclosed methods and compositions. In two separate water baths using glass beakers and/or vials, 95 grams of Protopet 1S petrolatum was heated to 32-33° C. and 5 grams of the VSV-MARV GP vaccine formulation was heated to 35-36° C. The heated petrolatum composition was homogenized using a spiral bit homogenizing rod while at 32-33° C. The homogenizing rod was placed in the center of the petrolatum and run at 600-1100 rpm until the petrolatum exhibits uniform viscosity. The heated vaccine formulation was then combined with the homogenized petrolatum either while the homogenizing rod is still running or while the homogenizing rod is turned off. Immediately following the addition of the heated vaccine formulation to the petrolatum composition, the homogenizer was turned back on and run at 600-1100 rpm for one minute in order to achieve uniform viscosity. The mixed and homogenized composition was then allowed to cool to room temperature.

### Example 22—Formulation Efficacy and Evaluation

The formulation prepared in Example 21 was tested to determine formulation efficacy. The vaccine formulation was placed on a hot plate and was mixed with a sterile spatula to ensure the composition was heated evenly and showed a uniform consistency. The vaccine formulation was brought to a temperature of ˜35.6° C. then removed from the heat source and mixed using an OLED digital overhead stirrer (˜650 rpm) until the composition was uniform. Mixing was paused momentarily, and the composition was inoculated with a 5 mL volume of the test virus maintained at 36.0° C.±1° C. (to ensure test virus was warmer than the test substance). The test substance was at ˜34° C. when inoculated with the test virus. The test substance/virus mixture was held for each hold time specified by the Study Sponsor at an exposure temperature of room temperature. Then to end each hold time, a sample was extracted/harvested, transferred to a sterile conical, and weighed out (0.700 g sample mixture). Then a 6.3 mL volume of sterile DI water (containing sterile glass beads) was added to the 0.700 g sample mixture (this was considered the 10-2 dilution) and serially diluted out to the appropriate dilution. A recovery control was performed alongside the test with DI water instead and treated in a similar manner. The recovery control was held for the Study Sponsor specified hold times at the Study Sponsor specified exposure temperature, harvested, and serially diluted to the appropriate dilution. Then to facilitate virus-host cell adsorption, an adsorption step was performed by inoculating the dilutions into the host cell cultures which did not contain test medium. The assay plates were incubated at 36±2° C. in a humidified atmosphere of 6±1% CO2 for 60 minutes. Following adsorption, each well received a 3 mL aliquot of agarose overlay and returned to the incubator for the remainder of the incubation period. The inoculated cell culture plates were incubated for the period most suitable for the virus-host cell system (e.g. ˜48 hours). Following the incubation period, the assay was assessed for plaque formation and results read via crystal violet staining (performed per the procedure provided by IAVI). Log 10 calculations were computed for test sample mixture relative to the control virus mixture and reported to the Study Sponsor. Per each day of testing, a cold virus control was performed. A 5 mL volume of virus (maintained at 2-8° C.) was inoculated into 95 mL of sterile DI water (maintained at room temperature). Vesicular stomatitis control virus was tested as propagated (virus contained 2% fetal bovine serum (FBS) organic soil load). To keep the control virus's titer similar to that of the test virus, control virus was not diluted. The Infectious Titer (PFU/mL) was calculated as: # of plaques×dilution factor×2. The result of this calculation is expressed as PFU/mL (or volume of dilution inoculated) for the test vaccine composition and virus controls. The results of the study are shown in Tables 1-4.

TABLE 1
Cold Control Virus Results

| Cell Control | Healthy Monolayer | Healthy Monolayer |
|---|---|---|
| 10⁻³ Dilution | N/A | TNTC |
| 10⁻⁴ Dilution | N/A | TNTC |
| 10⁻⁵ Dilution | TNTC | TNTC |
| 10⁻⁶ Dilution | 39 | 23 |
| 10⁻⁷ Dilution | 0 | 1 |
| 10⁻⁸ Dilution | 0 | 0 |
| 10⁻⁹ Dilution | 0 | 0 |
| PFU/mL | 7.89 Log₁₀ | 7.66 Log₁₀ |

TNTC = Too Numerous to Count

TABLE 2
Recovery Control Results for VSV in DI Water

| Contact Time | 10 Minutes | 2 Hours | 24 Hours |
|---|---|---|---|
| 10⁻² Dilution | TNTC | TNTC | TNTC |
| 10⁻³ Dilution | TNTC | TNTC | TNTC |
| 10⁻⁴ Dilution | TNTC | TNTC | TNTC |
| 10⁻⁵ Dilution | 36.5 | 28 | 40.5 |
| 10⁻⁶ Dilution | 4.5 | 4.5 | 12.5 |
| 10⁻⁷ Dilution | 0 | 0 | 1.5 |
| 10⁻⁸ Dilution | 0 | 0 | 0 |
| 10⁻⁹ Dilution | 0 | 0 | N/A |
| PFU/mL | 6.86 Log₁₀ | 6.75 Log₁₀ | 6.91 Log₁₀ |

TNTC = Too Numerous to Count

TABLE 3
Test Results for Sonneborn White Protopet 1S with
VSV-MARV-GP Vaccine VPN1011 15% Trehalose in HBSS

| Contact Time | 10 Minutes | 2 Hours | 24 Hours |
|---|---|---|---|
| 10⁻² Dilution | TNTC | TNTC | TNTC |
| 10⁻³ Dilution | TNTC | TNTC | 9.5* |
| 10⁻⁴ Dilution | 25 | TNTC | 0.5 |
| 10⁻⁵ Dilution | 7.5* | 3.5* | 0 |
| 10⁻⁶ Dilution | 0 | 0 | 0 |
| 10⁻⁷ Dilution | 0 | 0 | 0 |
| 10⁻⁸ Dilution | 0 | 0 | 0 |
| 10⁻⁹ Dilution | 0 | 0 | N/A |
| PFU/mL | 5.70 Log₁₀ | 5.85 Log₁₀ | 4.28 Log₁₀ |

TNTC = Too Numerous to Count
*Difficult to read as plaques merged together, merged plaques counted as one; plaque counts are estimated.

TABLE 4
Testing Conditions

| | | | Temperature | Relative | CO₂ |
|---|---|---|---|---|---|
| | | | (° C.) | Humidity | Conditions |
| Parameter | Duration | | Start/End | (%) | (%) |
| Treatment: | 10 | minutes | 22.5/22.5 | 42/43 | N/A |
| Sonneborn White | 2 | hours | 22.5/21.9 | 42/44 | N/A |
| Protopet 1S | 24 | hours | 22.5/21.8 | 42/45 | N/A |
| Incubation of | ~48 | hours | 36.0/36.0 | N/A | 6.0/6.0 |
| Assay Plates | | | | | |
| (10 min and 2 | | | | | |
| hr test/control | | | | | |
| plates) | | | | | |
| Incubation of | ~48 | hours | 36.0/36.0 | N/A | 6.0/6.0 |
| Assay Plates (24 | | | | | |
| hr test/control | | | | | |
| plates) | | | | | |

The results shown in Tables 1-4 indicate that the VSV vaccine formulation prepared in Example 21 maintains efficacy when combined and formulated with petrolatum according to the presently disclosed methods.

### Example 23—Novel Vaccine Viability Assay

Virucidal stability of the presently disclosed compositions and formulations was determined using an assay that involved three different dilution schemes. Vesicular stomatitis virus, Indiana strain, ATCC VR-158 was used in the assay and titrated via TCID₅₀ to evaluate the viability of the virus in petrolatum after two different hold times based on an estimated virus titer concentration of 8.50 log₁₀. “Dilution Scheme 1” involved 0.400 mL of virus to 3.6 g petrolatum and diluting with 36 mL of mineral oil. “Dilution Scheme 2” involved 0.200 mL of virus to 1.8 g petrolatum and diluting with 18 mL of mineral oil. “Dilution Scheme 3” involved 0.100 mL of virus to 0.90 g petrolatum and diluting with 9 mL of mineral oil.

An aliquot of virus was inoculated into an aliquot of HBSS. The test/control viruses were placed in a water bath set to 36±1° C. to equilibrate for at least 10 minutes. The test substance was placed on a hot plate and was mixed with a sterile spatula to ensure the substance was heated evenly and showed a uniform consistency. The test was brought to a temperature of −36.0° C. then removed from the heat source and mixed until the compound was uniform. Mixing was paused momentarily, and the compound was inoculated with a certain volume of the test virus maintained at 36.0±1° C., to ensure that the test virus was warmer than the test substance, and then mixed thoroughly for at least 1 minute. The test substance was at −34° C. when inoculated with the test virus then it was held for the specified hold time.

At the end of the hold time, an aliquot was removed and the mineral oil with sterile glass beads added to the mixture and vortexed. This served as the first dilution. Subsequent 10-fold serial dilutions were performed in 2% FBS DMEM, with the first dilution in mineral oil. Sterile glass beads were added to the first dilution tube to aid in homogenization. Virus, cytotoxicity, and cell culture controls were run alongside the test. Virus was plated by directly inoculating on to host cell. Cytotoxicity was performed for the petrolatum and mineral oil, with no virus inoculated. The host cell culture control had no inoculation of virus or sample(s). All dilutions were plated in quadruplicate onto 24-well plates prepared with Vero host cell line monolayer at an appropriate confluency. Virus aliquots were inoculated into plates without media, placed on an orbital rotator, and incubated for at least 30 minutes. Host cell controls were inoculated with media instead. Following the absorption, each well received 1.0 mL aliquot of test medium (e.g., 2% FBS EMEM). Plates were incubated under suitable conditions for virus amplification and detection. Cultures were observed for cytotoxicity approximately 24 hours post-inoculation, with media replaced if necessary. Plates were periodically checked for cytopathic effect, cytotoxicity, and contamination. On the final day, wells were scored microscopically for viral cytopathic effect, cytotoxic effects, and contamination using the TCID₅₀ Spearman-Karber method. The TCID₅₀/0.1 mL was determined, and log and percent reduction were calculated.

The Spearman-Karber method was calculated as follows: Negative logarithm of endpoint titer=[−Log of first dilution inoculated]−[((sum of % mortality at each dilution/100)−0.5)×Logarithm of dilution]. The result of this calculation is expressed in TCID₅₀/volume of dilution inoculated (e.g., 0.1 mL) for the test and recovery control. The log₁₀ reduction level was calculated as follows: Virus Control Suspension TCID₅₀−Virus-Substance Test Suspension TCID₅₀. The percent reduction level was calculated as follows: % Reduction=1−(C/B)×100, where: B=TCID₅₀ of virus control suspensions and C=TCID₅₀ of virus in virus-test suspensions. The results of this assay are shown in Tables 5-9. In Tables 5-9, “+”=Virus Recovered; “0”=Virus Not Recovered and/or No Cytotoxicity Observed; “T”=Cytotoxicity Observed.

TABLE 5
Test Results - 24 Hour Hold Time Test Results

24 Hour Hold Time Test Results

| | Dilution | Dilution | Dilution |
|---|---|---|---|
| Dilution | Scheme 1 | Scheme 2 | Scheme 3 |
| 10⁻² | + + + + | + + + + | + + + + |
| 10⁻³ | + + + + | + + + + | + + + + |
| 10⁻⁴ | + + + + | + + + + | + + + + |
| 10⁻⁵ | + + + + | + + + + | + + + + |
| 10⁻⁶ | + + + + | + + + + | + + + + |
| 10⁻⁷ | 0 + 0 + | + + + + | + + + + |
| TCID₅₀ per 0.1 mL | 7.00 log₁₀ | ≥7.50 log₁₀ | ≥7.50 log₁₀ |

TABLE 6
Test Results - 24 Hour Hold Time Control Results

24 Hour Hold Time Control Results

| | Dilution | Dilution | Dilution |
|---|---|---|---|
| Dilution | Scheme 1 | Scheme 2 | Scheme 3 |
| 10⁻² | + + + + | + + + + | + + + + |
| 10⁻³ | + + + + | + + + + | + + + + |
| 10⁻⁴ | + + + + | + + + + | + + + + |
| 10⁻⁵ | + + + + | + + + + | + + + + |
| 10⁻⁶ | + + + + | + + + + | + + + + |
| 10⁻⁷ | + 0 0 + | + + + + | + + + + |
| TCID₅₀ per 0.1 mL | 7.00 log₁₀ | ≥7.50 log₁₀ | ≥7.50 log₁₀ |

TABLE 7
Test Results - 72 Hour Hold Time Test Results

72 Hour Hold Time Test Results

| | Dilution | Dilution | Dilution |
|---|---|---|---|
| Dilution | Scheme 1 | Scheme 2 | Scheme 3 |
| 10⁻² | + + + + | + + + + | + + + + |
| 10⁻³ | + + + + | + + + + | + + + + |
| 10⁻⁴ | + + + + | + + + + | + + + + |
| 10⁻⁵ | + + + + | + + + + | + + + + |
| 10⁻⁶ | + + + + | + + + + | + + + + |
| 10⁻⁷ | + + + + | 0 0 + 0 | + + + + |
| TCID₅₀ per 0.1 mL | ≥7.50 log₁₀ | 6.75 log₁₀ | ≥7.50 log₁₀ |

TABLE 8
Test Results - 72 Hour Hold Time Control Results

24 Hour Hold Time Control Results

| | Dilution | Dilution | Dilution |
|---|---|---|---|
| Dilution | Scheme 1 | Scheme 2 | Scheme 3 |
| 10⁻² | + + + + | + + + + | + + + + |
| 10⁻³ | + + + + | + + + + | + + + + |
| 10⁻⁴ | + + + + | + + + + | + + + + |
| 10⁻⁵ | + + + + | + + + + | + + + + |
| 10⁻⁶ | + + + + | + + + + | + + + + |
| 10⁻⁷ | 0 0 + + | + + + + | + + + + |
| TCID₅₀ per 0.1 mL | 7.00 log10 | ≥7.50 log10 | ≥7.50 log10 |

TABLE 9
Test Results - 24 Hour Hold Time Controls

Test Control Results

| Dilution | Petrolatum | Mineral Oil |
|---|---|---|
| Cell Control | 0 0 0 0 | 0 0 0 0 |
| 10⁻¹ | 0 0 0 0 | 0 0 0 0 |
| 10⁻² | 0 0 0 0 | 0 0 0 0 |
| 10⁻³ | 0 0 0 0 | 0 0 0 0 |
| TCID₅₀ per 0.1 mL | ≤0.50 log₁₀ | ≤0.50 log₁₀ |

### Example 24—Virucidal Stability Tests

Virucidal stability of the presently disclosed compositions and formulations was determined using an assay that involved Vesicular stomatitis virus, Indiana strain, ATCC VR-158 and Vero Cells ATCC CCL-81 as the host cell line. Hold times were 3 days, 7 days, 14 days, and 28 days.

The test/control viruses were placed in a water bath set to 36±1° C. to equilibrate for at least 10 minutes. The test substance was placed on a hot plate and was mixed with a sterile spatula to ensure the substance was heated evenly and showed a uniform consistency. The test was brought to a temperature of ˜36.0° C. then removed from the heat source and mixed until the compound was uniform. Mixing was paused momentarily, and the compound was inoculated with 0.100 mL of the test virus maintained at 36.0±1° C., to ensure that the test virus was warmer than the test substance, and then mixed thoroughly for at least 1 minute. The test substance was at ˜34° C. when inoculated with the test virus then it was held for the specified hold time.

At the end of the hold time±1 hour, a 9 mL aliquot of mineral oil with sterile glass beads was added to the mixture and vortexed. That served as the second dilution, subsequent 10-fold serial dilutions were performed in 2% FBS EMEM, with the second dilution in mineral oil. The sterile glass beads were added to the second dilution tube to aid in homogenization. Virus, cytotoxicity, and cell culture controls were run alongside the test. Virus was plated by directly inoculating on to host cell. Cytotoxicity was performed for the petrolatum and mineral oil, with no virus inoculated. The host cell culture control had no inoculation of virus or sample(s). All dilutions were plated in quadruplicate onto 24-well plates prepared with Vero host cell line monolayer at an appropriate confluency. Virus aliquots were inoculated into plates without media, placed on an orbital rotator, and incubated for at least 30 minutes. Host cell controls were inoculated with media instead. Following the absorption, each well received 1.0 mL aliquot of test medium (e.g., 2% FBS EMEM). Plates were incubated under suitable conditions for virus amplification and detection. Cultures were observed for cytotoxicity approximately 24 hours post-inoculation, with media replaced if necessary. Plates were periodically checked for cytopathic effect, cytotoxicity, and contamination. On the final day, wells were scored microscopically for viral cytopathic effect, cytotoxic effects, and contamination using the TCID₅₀ Spearman-Karber method. The TCID₅₀/0.1 mL was determined, and log and percent reduction were calculated.

The Spearman-Karber method was calculated as follows: Negative logarithm of endpoint titer=[−Log of first dilution inoculated]−[((sum of % mortality at each dilution/100)−0.5)×Logarithm of dilution]. The result of this calculation is expressed in TCID₅₀/volume of dilution inoculated (e.g., 0.1 mL) for the test and recovery control. The log₁₀ reduction level was calculated as follows: Virus Control Suspension TCID₅₀−Virus-Substance Test Suspension TCID₅₀. The percent reduction level was calculated as follows: % Reduction=1−(C/B)×100, where: B=TCID₅₀ of virus control suspensions and C=TCID₅₀ of virus in virus-test suspensions. The results of this assay are shown in Tables 10-15. In Tables 10-15, “+”=Virus Recovered; “0”=Virus Not Recovered and/or No Cytotoxicity Observed; “T”=Cytotoxicity Observed. Based on the results shown in Tables 10-15, the presently disclosed thermostable mucosal vaccine compositions are expected to maintain potency and be medically effective when stored at a temperature of about 4 degrees Celsius for 28 days or after exposed to ambient temperatures for 14 days following removal from temperature-controlled storage.

TABLE 10
3-Day Harvest Results

| | Test Replicate | | | | | |
|---|---|---|---|---|---|---|
| | MARV | MARV | MARV | MARV | | |
| | in | in | in | in | IND in | IND in |
| | Petro- | Petro- | 2% FBS | 2% FBS | 2% FBS | 2% FBS |
| Dilu- | latum | latum | EMEM | EMEM | EMEM | EMEM |
| tion | (ambient) | (4° C.) | (ambient) | (4° C.) | (ambient) | (4° C.) |
| 10⁻² | + + + + | + + + + | + + + + | + + + + | + + + + | + + + + |
| 10⁻³ | + + + + | + + + + | + + + + | + + + + | + + + + | + + + + |
| 10⁻⁴ | + + + + | + + + + | + + + + | + + + + | + + + + | + + + + |
| 10⁻⁵ | + + + + | + + + + | + + + + | + + + + | + + + + | + + + + |
| 10⁻⁶ | + + + + | + + + + | + + + + | + + + + | + + + + | + + + + |
| 10⁻⁷ | + + + + | + + + + | + + + + | + + + + | + + + + | + + + + |
| TCID₅₀ | ≥7.50 | ≥7.50 | ≥7.50 | ≥7.50 | ≥7.50 | ≥7.50 |
| per 0.1 | | | | | | |
| mL | | | | | | |

TABLE 11
7-Day Harvest Results

| | Test Replicate | | | | | |
|---|---|---|---|---|---|---|
| | MARV | MARV | MARV | MARV | | |
| | in | in | in | in | IND in | IND in |
| | Petro- | Petro- | 2% FBS | 2% FBS | 2% FBS | 2% FBS |
| Dilu- | latum | latum | EMEM | EMEM | EMEM | EMEM |
| tion | (ambient) | (4° C.) | (ambient) | (4° C.) | (ambient) | (4° C.) |
| 10⁻² | + + + + | + + + + | + + + + | + + + + | + + + + | + + + + |
| 10⁻³ | + + + + | + + + + | + + + + | + + + + | + + + + | + + + + |
| 10⁻⁴ | + + + + | + + + + | + + + + | + + + + | + + + + | + + + + |
| 10⁻⁵ | + + + + | + + + + | + + + + | + + + + | + + + + | + + + + |
| 10⁻⁶ | + + + + | + + + + | + + + + | + + + + | + + + + | + + + + |
| 10⁻⁷ | 0 + 0 0 | + + + + | + + + + | + + + + | + + + + | + + + + |
| TCID₅₀ | 6.75 | ≥7.50 | ≥7.50 | ≥7.50 | ≥7.50 | ≥7.50 |
| per 0.1 | | | | | | |
| mL | | | | | | |

TABLE 12
14-Day Harvest Results

| | Test Replicate | | | | | |
|---|---|---|---|---|---|---|
| | MARV | MARV | MARV | MARV | | |
| | in | in | in | in | IND in | IND in |
| | Petro- | Petro- | 2% FBS | 2% FBS | 2% FBS | 2% FBS |
| Dilu- | latum | latum | EMEM | EMEM | EMEM | EMEM |
| tion | (ambient) | (4° C.) | (ambient) | (4° C.) | (ambient) | (4° C.) |
| 10⁻² | + + + + | + + + + | + + + + | + + + + | + + + + | + + + + |
| 10⁻³ | + + + + | + + + + | + + + + | + + + + | + + + + | + + + + |
| 10⁻⁴ | + + + + | + + + + | + + + + | + + + + | + + + + | + + + + |
| 10⁻⁵ | + + + + | + + + + | + + + + | + + + + | + + + + | + + + + |
| 10⁻⁶ | 0 + 0 0 | + + + + | + + + + | + + + + | + + + + | + + + + |
| 10⁻⁷ | 0 0 0 + | + + + + | + + + 0 | + + + + | + + + 0 | + + + + |
| TCID₅₀ | 6.00 | ≥7.50 | 7.25 | ≥7.50 | 7.25 | ≥7.50 |
| per 0.1 | | | | | | |
| mL | | | | | | |

TABLE 13
28-Day Harvest Results

| | Test Replicate | | | | | |
|---|---|---|---|---|---|---|
| | MARV | MARV | MARV | MARV | | |
| | in | in | in | in | IND in | IND in |
| | Petro- | Petro- | 2% FBS | 2% FBS | 2% FBS | 2% FBS |
| Dilu- | latum | latum | EMEM | EMEM | EMEM | EMEM |
| tion | (ambient) | (4° C.) | (ambient) | (4° C.) | (ambient) | (4° C.) |
| 10⁻² | + + + + | + + + + | + + + + | + + + + | + + + + | + + + + |
| 10⁻³ | + + + + | + + + + | + + + + | + + + + | + + + + | + + + + |
| 10⁻⁴ | + + + + | + + + + | + + + + | + + + + | + + + + | + + + + |
| 10⁻⁵ | 0 0 0 0 | + + + + | 0 0 0 0 | + + + + | + + + + | + + + + |
| 10⁻⁶ | 0 0 0 0 | + + + + | 0 0 0 0 | + + + + | 0 0 0 0 | + + + + |
| 10⁻⁷ | 0 0 0 0 | + + + + | 0 0 0 0 | + + + + | 0 0 0 0 | 0 0 + + |
| TCID₅₀ | 4.50 | ≥7.50 | 4.50 | ≥7.50 | 5.50 | 7.00 |
| per 0.1 | | | | | | |
| mL | | | | | | |

TABLE 14
Controls for Each Harvest Date

| | | Harvest Day | | | |
|---|---|---|---|---|---|
| Parameter | Dilution | 3-Day | 7-Day | 14-Day | 28-Day |
| Cell Control | N/A | 0 0 0 0 | 0 0 0 0 | 0 0 0 0 | 0 0 0 0 |
| Cytotoxicity Control | 10⁻¹ | 0 0 0 0 | N/A | N/A | N/A |
| Petrolatum | 10⁻² | 0 0 0 0 | | | |
| | 10⁻³ | 0 0 0 0 | | | |
| Cytotoxicity Control | 10⁻¹ | 0 0 0 0 | N/A | N/A | N/A |
| Mineral Oil | 10⁻² | 0 0 0 0 | | | |
| | 10⁻³ | 0 0 0 0 | | | |

TABLE 15
Testing Conditions

| | | Temperature (° C.) | | Relative |
|---|---|---|---|---|
| | | Start/End | | Humidity (%) |
| Parameter | Duration | Ambient | 4° C. | Start/End |
| Hold Time: 3 Days | ~3 days | 22.0/21.5 | 5.3/5.2 | 42/45 |
| Hold Time: 7 Days | ~7 days | 22.0/21.7 | 5.3/5.0 | 42/43 |
| Hold Time: 14 Days | ~14 days | 22.1/22.8 | 5.1/5.0 | 43/51 |
| Hold Time: 28 Days | ~28 days | 22.2/23.9 | 5.3/7.2 | 44/49 |

| | | | |
|---|---|---|---|
| Incubation of Assay | ~7 days | 35.4/36.0 | 6.0/6.0 |
| Plates 3 Days | | | (% CO₂) |
| Incubation of Assay | ~7 days | 35.6/35.6 | 6.0/6.0 |
| Plates 7 Days | | | (% CO₂) |
| Incubation of Assay | ~7 days | 35.6/36.0 | 6.0/6.0 |
| Plates 14 Days | | | (% CO₂) |
| Incubation of Assay | ~7 days | 35.8/36.1 | 6.0/6.0 |
| Plates 28 Days | | | (% CO₂) |

Other objects, features and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein.

### Statements of the Disclosure:

Statement 1. A method of stabilizing a vaccine composition for mucosal use, the method comprising: combining a vaccine composition for mucosal use with a petrolatum carrier.

Statement 2. A method of increasing the stability of a vaccine composition for mucosal use during storage or prior to administration to a subject in need thereof, the method comprising: combining a vaccine composition for mucosal use with a petrolatum carrier.

Statement 3. A method of increasing the resistance to potency loss of a vaccine composition for mucosal use during storage or prior to administration to a subject in need thereof, the method comprising: combining a vaccine composition for mucosal use with a petrolatum carrier.

Statement 4. A method of increasing the time period that a vaccine composition for mucosal use may be stored prior to administration to a subject in need thereof without a substantial loss in potency or effectiveness, the method comprising: combining a vaccine composition for mucosal use with a petrolatum carrier.

Statement 5. A method of stabilizing a vaccine composition for mucosal use during temperature excursions during storage or during the period of time between removal of the vaccine composition from temperature-controlled storage and administration of the vaccine composition to a subject, the method comprising: combining a vaccine composition for mucosal use with a petrolatum carrier.

Statement 6. A method of protecting a vaccine composition from potency loss resulting from temperature excursions during storage or during the period of time between removal of the vaccine composition from storage and administration of the vaccine composition to a subject, the method comprising: combining a vaccine composition for mucosal use with a petrolatum carrier.

Statement 7. A method of preparing a thermostable vaccine composition for mucosal use, the method comprising: combining a vaccine composition with a petrolatum carrier.

Statement 8. The method according to any one of Statements 1-7, wherein the vaccine composition is a liquid vaccine composition.

Statement 9. The method according to any one of Statements 1-7, wherein the vaccine composition comprises at least one antigen or antigen-encoding composition.

Statement 10. The method according to any one of Statements 1-7, wherein the vaccine composition is a liquid comprising at least one antigen.

Statement 11. The method according to any one of Statements 1-7, wherein the vaccine composition is a mRNA vaccine composition.

Statement 12. The method according to any one of Statements 1-7, wherein the vaccine composition is selected from the group consisting of: live attenuated vaccine (LAV) compositions, inactivated vaccine compositions, subunit vaccine compositions, conjugate vaccine compositions, toxoid vaccine compositions, mRNA vaccine compositions, viral vector vaccine compositions.

Statement 13. The method according to any one of Statements 1-12, wherein the thermostable vaccine composition is operable to maintain at least 50% potency when stored at a temperature of from about 2 degrees Celsius to about 8 degrees Celsius for 12 months.

Statement 14. The method according to any one of Statements 1-12, wherein the thermostable vaccine composition is operable to maintain at least 80% potency when exposed to ambient temperatures for 24 hours following removal from temperature-controlled storage.

Statement 15. The method according to any one of Statements 1-14, wherein combining the vaccine composition with the petrolatum carrier forms a stabilized mucosal vaccine, the method further comprising: storing the stabilized mucosal vaccine at a temperature of from about 2 degrees Celsius to about 8 degrees Celsius for a predetermined period of time prior to administration to the subject.

Statement 16. The method according to Statement 15, wherein the predetermined period of time is greater than 30 days, or greater than 60 days, or greater than 90 days, or greater than 120 days, or at least 3 months, or greater than 6 hours, or greater than 8 hours, or greater than 10 hours, or greater than 12 hours, or greater than 24 hours.

Statement 17. The method according to any one of Statements 1-14, wherein combining the vaccine composition with a petrolatum carrier forms a stabilized mucosal vaccine, the method further comprising: storing the stabilized mucosal vaccine at a temperature of from about 8.5 degrees Celsius to about 25 degrees Celsius for a predetermined period of time prior to administration to the subject.

Statement 18. The method according to Statement 17, wherein the predetermined period of time is greater than 2 hours or greater than 6 hours.

Statement 19. The method according to Statement 17, wherein the predetermined period of time is greater than 12 hours.

Statement 20. The method according to any one of Statements 1-14, wherein combining the vaccine composition with a petrolatum carrier forms a stabilized mucosal vaccine, the method further comprising: storing the stabilized mucosal vaccine at a temperature of from about 20 degrees Celsius to about 25 degrees Celsius for a predetermined period of time prior to administration to the subject.

Statement 21. The method according to Statement 20, wherein the predetermined period of time is greater than 2 hours.

Statement 22. The method according to Statement 20, wherein the predetermined period of time is greater than 6 hours.

Statement 23. The method according to Statement 20, wherein the predetermined period of time is greater than 12 hours.

Statement 24. A method of increasing the stability of a liquid vaccine for mucosal use during temperature excursions associated with cold storage of the vaccine, the method comprising: combining a liquid comprising at least one antigen or antigen encoding composition with a petrolatum carrier.

Statement 25. A method of increasing the resistance to potency loss of a liquid vaccine for mucosal use during temperature excursions associated with cold storage of the vaccine, the method comprising: combining a liquid comprising at least one antigen or antigen encoding composition with a petrolatum carrier.

Statement 26. A method of increasing the time period that a liquid vaccine for mucosal use may be stored in a cold storage refrigeration unit prior to administration to a subject in need thereof without a substantial loss in potency or effectiveness, the method comprising: combining a liquid comprising at least one antigen or antigen encoding composition with a petrolatum carrier.

Statement 27. The method according to Statement 26, wherein the cold storage refrigeration unit is set to maintain a temperature of from about 2 degrees Celsius to about 8 degrees Celsius.

Statement 28. The method according to Statement 26, wherein the cold storage refrigeration unit is designed to maintain a temperature of from about 2 degrees Celsius to about 8 degrees Celsius.

Statement 29. The method according to any one of Statements 24-28, wherein combining a liquid comprising at least one antigen or antigen encoding composition with a petrolatum carrier forms a stabilized mucosal vaccine, the method further comprising: storing the stabilized mucosal vaccine in a cold storage refrigeration unit for a predetermined amount of time prior to administration to the subject, the unit set to maintain a temperature within the unit of from 2 degrees Celsius to 8 degrees Celsius.

Statement 30. The method according to Statement 29, wherein the predetermined period of time is greater than 30 days.

Statement 31. The method according to Statement 29, wherein the predetermined period of time is greater than 60 days.

Statement 32. The method according to Statement 29, wherein the predetermined period of time is greater than 90 days.

Statement 33. The method according to Statement 29, wherein the predetermined period of time is greater than 120 days.

Statement 34. The method according to Statement 29, wherein the predetermined period of time is at least 3 months.

Statement 35. The method according to Statement 29, wherein the predetermined period of time is greater than 6 hours.

Statement 36. The method according to Statement 29, wherein the predetermined period of time is greater than 8 hours.

Statement 37. The method according to Statement 29, wherein the predetermined period of time is greater than 10 hours.

Statement 38. The method according to Statement 29, wherein the predetermined period of time is greater than 12 hours.

Statement 39. The method according to Statement 29, wherein the predetermined period of time is greater than 24 hours.

Statement 40. A method of increasing the resistance to potency loss of a liquid vaccine for mucosal use during temporary storage of the vaccine at room temperature prior to administration of the vaccine to a subject, the method comprising: combining a liquid comprising at least one antigen or antigen encoding composition with a petrolatum carrier.

Statement 41. A method of increasing the time period that a liquid vaccine for mucosal use may be temporarily stored at room temperature prior to administration to a subject in need thereof without a substantial loss in potency or effectiveness, the method comprising: combining a liquid comprising at least one antigen or antigen encoding composition with a petrolatum carrier.

Statement 42. The method according to Statement 40 or Statement 41, wherein combining a liquid comprising at least one antigen or antigen encoding composition with a petrolatum carrier forms a stabilized mucosal vaccine, the method further comprising: storing the stabilized mucosal vaccine at a temperature of from about 20 degrees Celsius to about 25 degrees Celsius for a predetermined period of time prior to administration to the subject.

Statement 43. The method according to Statement 42, wherein the predetermined period of time is greater than 2 hours.

Statement 44. The method according to Statement 42, wherein the predetermined period of time is greater than 6 hours.

Statement 45. The method according to Statement 42, wherein the predetermined period of time is greater than 12 hours.

Statement 46. A method of increasing the resistance to potency loss of a liquid vaccine for mucosal use during temporary storage of the vaccine at ambient temperatures of from about 8.5 degrees Celsius to about 25 degrees Celsius prior to administration of the vaccine to a subject, the method comprising: combining a liquid comprising at least one antigen or antigen encoding composition with a petrolatum carrier.

Statement 47. A method of increasing the time period that a liquid vaccine for mucosal use may be temporarily stored at ambient temperatures of from about 8.5 degrees Celsius to about 25 degrees Celsius prior to administration to a subject in need thereof without a substantial loss in potency or effectiveness, the method comprising: combining a liquid comprising at least one antigen or antigen encoding composition with a petrolatum carrier.

Statement 48. The method according to Statement 46 or Statement 47, wherein combining a liquid comprising at least one antigen or antigen encoding composition with a petrolatum carrier forms a stabilized mucosal vaccine, the method further comprising: storing the stabilized mucosal vaccine at a temperature of from about 8.5 degrees Celsius to about 25 degrees Celsius for a predetermined period of time prior to administration to the subject.

Statement 49. The method according to Statement 48, wherein the predetermined period of time is greater than 2 hours.

Statement 50. The method according to Statement 48, wherein the predetermined period of time is greater than 6 hours.

Statement 51. The method according to Statement 48, wherein the predetermined period of time is greater than 12 hours.

Statement 52. A method of increasing the thermal stability of a liquid vaccine for muscosal use, the method comprising: combining a liquid comprising at least one antigen or antigen encoding composition with a petrolatum carrier.

Statement 53. A method of increasing the duration a liquid vaccine for muscosal use may be stored under ambient conditions, the method comprising: combining a liquid comprising at least one antigen or antigen encoding composition with a petrolatum carrier.

Statement 54. A method of increasing the dose transfer of a muscosal vaccine, the method comprising: combining a liquid comprising at least one antigen or antigen encoding composition with a petrolatum carrier.

Statement 55. A method of increasing the dosing predictability of a muscosal vaccine, the method comprising: combining a liquid comprising at least one antigen or antigen encoding composition with a petrolatum carrier.

Statement 56. A method of preparing a mucosal vaccine, the method comprising: combining a liquid comprising at least one antigen or antigen encoding composition with a petrolatum carrier to form the mucosal vaccine.

Statement 57. A method of preparing a mucosal vaccine with increased thermal stability, the method comprising: combining a liquid comprising at least one antigen or antigen encoding composition with a petrolatum carrier to form the mucosal vaccine.

Statement 58. A method of preparing a mucosal vaccine with increased dose transfer, the method comprising: combining a liquid comprising at least one antigen or antigen encoding composition with a petrolatum carrier to form the mucosal vaccine.

Statement 59. A method of preparing a mucosal vaccine with increased dosing predictability, the method comprising: combining a liquid comprising at least one antigen or antigen encoding composition with a petrolatum carrier to form the mucosal vaccine.

Statement 60. The method according to any one of Statements 1-59, wherein combining a vaccine composition or liquid comprising at least one antigen or antigen encoding composition with a petrolatum carrier forms a mucosal vaccine or a stabilized mucosal vaccine, the method further comprising administering the mucosal vaccine or stabilized mucosal vaccine to a mucosa or a mucosal membrane of a subject.

Statement 61. A method of vaccinating a subject, the method comprising: administering a petrolatum mucosal vaccine to a mucosa or a mucosal membrane of the subject, wherein the petrolatum mucosal vaccine comprises a liquid comprising at least one antigen or antigen encoding composition that has been combined with a petrolatum carrier.

Statement 62. A method of vaccinating a subject, the method comprising: combining a liquid comprising at least one antigen or antigen encoding composition with a petrolatum carrier to form a petrolatum mucosal vaccine; and administering the petrolatum mucosal vaccine to a mucosa or a mucosal membrane of the subject.

Statement 63. A method of causing an immune response in a subject, the method comprising: administering a petrolatum mucosal vaccine to a mucosa or a mucosal membrane of the subject, wherein the petrolatum mucosal vaccine comprises a liquid comprising at least one antigen or antigen encoding composition that has been combined with a petrolatum carrier.

Statement 64. A method of causing an immune response in a subject, the method comprising: combining a liquid comprising at least one antigen or antigen encoding composition with a petrolatum carrier to form a petrolatum mucosal vaccine; and administering the petrolatum mucosal vaccine to a mucosa or a mucosal membrane of the subject.

Statement 65. The method according to any one of Statements 61-64, wherein administering the petrolatum mucosal vaccine to the mucosa or a mucosal membrane of the subject comprises oral, nasal, rectal, or vaginal administration.

Statement 66. The method according to any one of Statements 61-64, wherein administering the petrolatum mucosal vaccine to the mucosa or a mucosal membrane of the subject comprises intranasal administration.

Statement 67. The method according to any one of Statements 1-66, wherein the liquid comprising at least one antigen or antigen encoding composition is a liquid vaccine suitable for mucosal administration.

Statement 68. The method according to any one of Statements 1-67, wherein the combining a vaccine composition or a liquid comprising at least one antigen or antigen encoding composition with a petrolatum carrier results in the liquid being suspended in the petrolatum carrier.

Statement 69. The method according to any one of Statements 1-68, wherein the combining a vaccine composition or a liquid comprising at least one antigen or antigen encoding composition with a petrolatum carrier comprises combining a liquid comprising at least one antigen or antigen encoding composition with a petrolatum carrier such that the liquid is suspended in the petrolatum carrier.

Statement 70. The method according to any one of Statements 1-69, wherein the combining a vaccine composition or a liquid comprising at least one antigen or antigen encoding composition with a petrolatum carrier comprises: a) providing a liquid comprising at least one antigen or antigen encoding composition; b) heating petrolatum or a composition comprising petrolatum to a temperature sufficient to cause the petrolatum to melt to give a melted petrolatum carrier composition; c) mixing the melted petrolatum carrier composition and the liquid comprising at least one antigen or antigen encoding composition to give a melted mixture; and d) cooling the melted mixture to provide a petrolatum mucosal vaccine composition.

Statement 71. The method according to any one of Statements 1-69, wherein the combining a vaccine composition or a liquid comprising at least one antigen or antigen encoding composition with a petrolatum carrier comprises: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) mixing the melted petrolatum carrier composition and the liquid comprising at least one antigen or antigen encoding composition to give a melted mixture; and d) allowing the melted mixture to cool to room temperature to provide a petrolatum mucosal vaccine composition.

Statement 72. The method according to any one of Statements 1-69, wherein the combining a vaccine composition or a liquid comprising at least one antigen or antigen encoding composition with a petrolatum carrier comprises: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) heating the liquid comprising at least one antigen or antigen encoding composition to a temperature equal to or less than the temperature of the melted petrolatum carrier composition to give a heated liquid antigen composition; d) mixing the melted petrolatum carrier composition and the heated liquid antigen composition to give a melted mixture; and e) allowing the melted mixture to cool to room temperature to provide a petrolatum mucosal vaccine composition.

Statement 73. The method according to any one of Statements 1-69, wherein the combining a vaccine composition or a liquid comprising at least one antigen or antigen encoding composition with a petrolatum carrier comprises: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) heating the liquid comprising at least one antigen or antigen encoding composition to a temperature that is about 1 degree C. to about 5 degrees Celsius higher than the temperature of the melted petrolatum carrier composition to give a heated liquid antigen composition; d) mixing the melted petrolatum carrier composition and the heated liquid antigen composition to give a melted mixture; and e) allowing the melted mixture to cool to room temperature to provide a petrolatum mucosal vaccine composition.

Statement 74. The method according to any one of Statements 1-73, further comprising: dissolving or combining at least one antigen or antigen encoding composition in a liquid to provide a liquid comprising at least one antigen or antigen encoding composition.

Statement 75. The method according to any one of Statements 1-74, wherein the liquid comprises one or more polar solvents.

Statement 76. The method according to Statement 75, wherein the liquid comprises water.

Statement 77. The method according to any one of Statements 1-76, wherein the liquid comprising at least one antigen or antigen encoding composition is suspended in the petrolatum carrier in the absence of an emulsifier or without the use of an added emulsifier.

Statement 78. The method according to any one of Statements 1-76, wherein the vaccine composition or the liquid comprising at least one antigen or antigen encoding composition is substantially suspended in the petrolatum.

Statement 79. The method according to any one of Statements 1-76, wherein the vaccine composition or liquid comprising at least one antigen or antigen encoding composition is dispersed in the petrolatum carrier in the absence of an emulsifier or without the use of an added emulsifier.

Statement 80. The method according to any one of Statements 1-76, wherein the vaccine composition or liquid comprising at least one antigen or antigen encoding composition is substantially dispersed in the petrolatum.

Statement 81. The method according to any one of Statements 1-76, wherein the vaccine composition or liquid comprising at least one antigen or antigen encoding composition is dispersed in the petrolatum carrier in the form of nanodroplets comprising the liquid and at least one antigen or antigen encoding composition without the use of an added emulsifier.

Statement 82. The method according to any one of Statements 1-76, wherein the vaccine composition or liquid comprising at least one antigen or antigen encoding composition is substantially dispersed in the petrolatum in the form of nanodroplets comprising the vaccine composition or liquid and at least one antigen or antigen encoding composition.

Statement 83. The method according to any one of Statements 1-82, wherein the liquid comprising at least one antigen or antigen encoding composition further comprises one or more preservatives.

Statement 84. The method according to Statement 83, wherein the one or more preservatives includes one or more cationic biocides.

Statement 85. The method according to Statement 84, wherein the one or more cationic biocides is selected from the group consisting of benzalkonium chloride, cetrimide, chlorhexidine, polihexanide biguanide, and any combination thereof.

Statement 86. The method according to Statement 84, wherein the one or more cationic biocides is a mixture of benzalkonium chloride and polihexanide biguanide.

Statement 87. The method according to Statement 84, wherein the one or more cationic biocides is benzalkonium chloride.

Statement 88. The method according to Statement 84, wherein the one or more cationic biocides is polihexanide biguanide.

Statement 89. The method according to any one of Statements 1-88, wherein the combining the vaccine composition or liquid comprising at least one antigen or antigen encoding composition with a petrolatum carrier forms a mucosal vaccine or a stabilized mucosal vaccine, the mucosal vaccine or stabilized mucosal vaccine comprising from about 10% by weight to about 90% by weight petrolatum carrier.

Statement 90. The method according to any one of Statements 1-88, wherein the combining the vaccine composition or liquid comprising at least one antigen or antigen encoding composition with a petrolatum carrier forms a mucosal vaccine or a stabilized mucosal vaccine, the mucosal vaccine or stabilized mucosal vaccine comprising from about 10% by weight to about 35% by weight petrolatum carrier.

Statement 91. The method according to any one of Statements 1-88, wherein the combining the vaccine composition or liquid comprising at least one antigen or antigen encoding composition with a petrolatum carrier forms a mucosal vaccine or a stabilized mucosal vaccine, the mucosal vaccine or stabilized mucosal vaccine comprising from about 40% by weight to about 60% by weight petrolatum carrier.

Statement 92. The method according to any one of Statements 1-88, wherein the combining the vaccine composition or liquid comprising at least one antigen or antigen encoding composition with a petrolatum carrier forms a mucosal vaccine or a stabilized mucosal vaccine, the mucosal vaccine or stabilized mucosal vaccine comprising from about 40% by weight to about 90% by weight petrolatum carrier.

Statement 93. The method according to any one of Statements 1-88, wherein the combining the vaccine composition or liquid comprising at least one antigen or antigen encoding composition with a petrolatum carrier forms a mucosal vaccine or a stabilized mucosal vaccine, the mucosal vaccine or stabilized mucosal vaccine comprising at least 80% by weight petrolatum carrier.

Statement 94. The method according to any one of Statements 1-88, wherein the combining the vaccine composition or liquid comprising at least one antigen or antigen encoding composition with a petrolatum carrier forms a mucosal vaccine or a stabilized mucosal vaccine, the mucosal vaccine or stabilized mucosal vaccine comprising at least 85% by weight petrolatum carrier.

Statement 95. The method according to any one of Statements 1-88, wherein the combining the vaccine composition or liquid comprising at least one antigen or antigen encoding composition with a petrolatum carrier forms a mucosal vaccine or a stabilized mucosal vaccine, the mucosal vaccine or stabilized mucosal vaccine comprising at least 90% by weight petrolatum carrier.

Statement 96. The method according to any one of Statements 1-95, wherein the vaccine composition or liquid comprising at least one antigen or antigen encoding composition comprises one or more components selected from the group consisting of lactose, sucrose, glycine, monosodium glutamate, human serum albumin, and bovine serum albumin.

Statement 97. The method according to any one of Statements 1-96, wherein the at least one antigen or antigen encoding composition is selected from the group consisting of a live attenuated vaccine (LAV), an inactivated (killed antigen) vaccine, a subunit (purified antigen) vaccine, a toxoid (inactivated toxin) vaccine, a bacteriophage, an antigen carried or delivered by a bacteriophage, a conjugate vaccine composition, a mRNA vaccine composition, a viral vector vaccine composition, an antigen carried or delivered by a recombinant vesicular stomatitis virus (rVSV) vector, and any combination thereof.

Statement 98. The method according to any one of Statements 1-97, wherein the at least one antigen or antigen encoding composition is operable, upon administration to a subject, to cause an immune response in a subject sufficient to prevent, mitigate, or treat one or more infections selected from the group consisting of Marburg Virus, Ebola Zaire, Ebola Sudan, Lassa Virus, Buruli Ulcers, Leprosy, SARS-CoV-2 virus, and any combination thereof.

Statement 99. The method according to any one of Statements 1-97, wherein the liquid comprising at least one antigen or antigen encoding composition is a liquid vaccine effective for the prevention, mitigation, or treatment of Marburg Virus, Ebola Zaire, Ebola Sudan, Lassa Fever, Buruli Ulcers, Leprosy, SARS-CoV-2 virus, and any combination thereof.

Statement 100. The method according to any one of Statements 1-99, wherein the liquid comprising at least one antigen or antigen encoding composition is a Marburg virus vaccine.

Statement 101. The method according to any one of Statements 1-99, wherein the liquid comprising at least one antigen or antigen encoding composition is a recombinant vesicular stomatitis virus (rVSV) Ebola vaccine.

Statement 102. The method according to any one of Statements 1-99, wherein the liquid comprising at least one antigen or antigen encoding composition is a recombinant vesicular stomatitis virus (rVSV) SARS-CoV-2 vaccine.

Statement 103. The method according to any one of Statements 1-99, wherein the liquid comprising at least one antigen or antigen encoding composition is a Lassa virus vaccine.

Statement 104. The method according to any one of Statements 1-99, wherein the liquid comprising at least one antigen or antigen encoding composition is a Leprosy vaccine.

Statement 105. The method according to any one of Statements 1-99, wherein the liquid comprising at least one antigen or antigen encoding composition is a Buruli Ulcer vaccine.

Statement 106. A thermostable mucosal vaccine composition comprising: a liquid comprising at least one antigen or antigen encoding composition; and a petrolatum carrier.

Statement 107. The composition according to Statement 106, wherein the thermostable mucosal vaccine composition is stable and effective when stored at a temperature of from about 2 degrees Celsius to about 8 degrees Celsius for a predetermined period of time prior to administration to a subject, wherein the predetermined period of time is greater than 6 hours.

Statement 108. The composition according to Statement 107, wherein the predetermined period of time is greater than 8 hours.

Statement 109. The composition according to Statement 107, wherein the predetermined period of time is greater than 10 hours.

Statement 110. The composition according to Statement 107, wherein the predetermined period of time is greater than 12 hours.

Statement 111. The composition according to Statement 107, wherein the predetermined period of time is greater than 24 hours.

Statement 112. The composition according to Statement 107, wherein the predetermined period of time is greater than 30 days.

Statement 113. The composition according to Statement 107, wherein the predetermined period of time is greater than 60 days.

Statement 114. The composition according to Statement 107, wherein the predetermined period of time is greater than 90 days.

Statement 115. The composition according to Statement 107, wherein the predetermined period of time is greater than 120 days.

Statement 116. The composition according to Statement 107, wherein the predetermined period of time is at least 3 months.

Statement 117. The composition according to Statement 106, wherein the thermostable mucosal vaccine composition is stable and effective when stored at a temperature of from about 8.5 degrees Celsius to about 25 degrees Celsius for a predetermined period of time prior to administration to a subject, wherein the predetermined period of time is greater than 2 hours.

Statement 118. The composition according to Statement 117, wherein the predetermined period of time is greater than 6 hours.

Statement 119. The composition according to Statement 117, wherein the predetermined period of time is greater than 12 hours.

Statement 120. The composition according to Statement 106, wherein the thermostable mucosal vaccine composition is stable and effective when stored at a temperature of from about 20 degrees Celsius to about 25 degrees Celsius for a predetermined period of time prior to administration to a subject, wherein the predetermined period of time is greater than 2 hours.

Statement 121. The composition according to Statement 120, wherein the predetermined period of time is greater than 6 hours.

Statement 122. The composition according to Statement 120, wherein the predetermined period of time is greater than 12 hours.

Statement 123. The composition according to any one of Statements 106-122, wherein the thermostable mucosal vaccine composition is formulated for oral, nasal, rectal, or vaginal administration.

Statement 124. The composition according to any one of Statements 106-122, wherein the thermostable mucosal vaccine composition is formulated for intranasal administration.

Statement 125. The composition according to any one of Statements 106-124, wherein the liquid comprising at least one antigen or antigen encoding composition is a liquid vaccine suitable for mucosal administration.

Statement 126. The composition according to any one of Statements 106-125, wherein the liquid is suspended in the petrolatum carrier.

Statement 127. The composition according to any one of Statements 106-125, wherein the liquid comprising at least one antigen or antigen encoding composition with a petrolatum carrier is combined such that the liquid is suspended in the petrolatum carrier.

Statement 128. The composition according to any one of Statements 106-127, wherein the thermostable mucosal vaccine composition is prepared by: a) providing a liquid comprising at least one antigen or antigen encoding composition; b) heating petrolatum or a composition comprising petrolatum to a temperature sufficient to cause the petrolatum to melt to give a melted petrolatum carrier composition; c) mixing the melted petrolatum carrier composition and the liquid comprising at least one antigen or antigen encoding composition to give a melted mixture; and d) cooling the melted mixture to provide a petrolatum vaccine composition or mucosal vaccine composition.

Statement 129. The composition according to any one of Statements 106-127, wherein the thermostable mucosal vaccine composition is prepared by: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) mixing the melted petrolatum carrier composition and the liquid comprising at least one antigen or antigen encoding composition to give a melted mixture; and d) allowing the melted mixture to cool to room temperature to provide a petrolatum mucosal vaccine composition.

Statement 130. The composition according to any one of Statements 106-127, wherein the thermostable mucosal vaccine composition is prepared by: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) heating the liquid comprising at least one antigen or antigen encoding composition to a temperature equal to or less than the temperature of the melted petrolatum carrier composition to give a heated liquid antigen composition; d) mixing the melted petrolatum carrier composition and the heated liquid antigen composition to give a melted mixture; and e) allowing the melted mixture to cool to room temperature to provide a petrolatum mucosal vaccine composition.

Statement 131. The composition according to any one of Statements 106-127, wherein the thermostable mucosal vaccine composition is prepared by: a) providing a liquid comprising at least one antigen or antigen encoding composition; b) heating petrolatum or a composition comprising petrolatum to a temperature of from about 37 degrees Celsius to about 40 degrees Celsius to give a melted petrolatum carrier composition; c) heating the liquid comprising at least one antigen or antigen encoding composition to a temperature that is about 1 degree C. to about 5 degrees Celsius higher than the temperature of the melted petrolatum carrier composition to give a heated liquid antigen composition; d) mixing the melted petrolatum carrier composition and the heated liquid antigen composition to give a melted mixture; and e) allowing the melted mixture to cool to room temperature to provide a petrolatum mucosal vaccine composition.

Statement 132. The composition according to any one of Statements 106-131, further comprising: dissolving or combining at least one antigen or antigen encoding composition in a liquid to provide a liquid comprising at least one antigen or antigen encoding composition.

Statement 133. The composition according to any one of Statements 106-132, wherein the liquid comprises one or more polar solvents.

Statement 134. The composition according to Statement 133, wherein the liquid comprises water.

Statement 135. The composition according to any one of Statements 106-134, wherein the liquid comprising at least one antigen or antigen encoding composition is suspended in the petrolatum carrier in the absence of an emulsifier or without the use of an added emulsifier.

Statement 136. The composition according to any one of Statements 106-135, wherein the liquid comprising at least one antigen or antigen encoding composition is substantially suspended in the petrolatum.

Statement 137. The composition according to any one of Statements 106-135, wherein the liquid comprising at least one antigen or antigen encoding composition is dispersed in the petrolatum carrier in the absence of an emulsifier or without the use of an added emulsifier.

Statement 138. The composition according to any one of Statements 106-135, wherein the liquid comprising at least one antigen or antigen encoding composition is substantially dispersed in the petrolatum.

Statement 139. The composition according to any one of Statements 106-135, wherein the liquid comprising at least one antigen or antigen encoding composition is dispersed in the petrolatum carrier in the form of nanodroplets comprising the liquid and at least one antigen or antigen encoding composition without the use of an added emulsifier.

Statement 140. The composition according to any one of Statements 106-135, wherein the liquid comprising at least one antigen or antigen encoding composition is substantially dispersed in the petrolatum in the form of nanodroplets comprising the liquid and at least one antigen or antigen encoding composition.

Statement 141. The composition according to any one of Statements 106-140, wherein the liquid comprising at least one antigen or antigen encoding composition further comprises one or more preservatives.

Statement 142. The composition according to Statement 141, wherein the one or more preservatives comprises sodium chloride.

Statement 143. The composition according to Statement 141, wherein the one or more preservatives includes one or more cationic biocides.

Statement 144. The composition according to Statement 143, wherein the one or more cationic biocides is selected from the group consisting of benzalkonium chloride, cetrimide, chlorhexidine, polihexanide biguanide, and any combination thereof.

Statement 145. The composition according to Statement 144, wherein the one or more cationic biocides is a mixture of benzalkonium chloride and polihexanide biguanide.

Statement 146. The composition according to Statement 144, wherein the one or more cationic biocides is benzalkonium chloride.

Statement 147. The composition according to Statement 144, wherein the one or more cationic biocides is polihexanide biguanide.

Statement 148. The composition according to any one of Statements 106-147, wherein the thermostable mucosal vaccine comprises from about 10% by weight to about 90% by weight petrolatum carrier.

Statement 149. The composition according to any one of Statements 106-147, wherein the thermostable mucosal vaccine comprises from about 10% by weight to about 35% by weight petrolatum carrier.

Statement 150. The composition according to any one of Statements 106-147, wherein the thermostable mucosal vaccine comprises from about 40% by weight to about 60% by weight petrolatum carrier.

Statement 151. The composition according to any one of Statements 106-147, wherein the thermostable mucosal vaccine comprises from about 40% by weight to about 90% by weight petrolatum carrier.

Statement 152. The composition according to any one of Statements 106-147, wherein the thermostable mucosal vaccine comprises at least 80% by weight petrolatum carrier.

Statement 153. The composition according to any one of Statements 106-147, wherein the thermostable mucosal vaccine comprises at least 85% by weight petrolatum carrier.

Statement 154. The composition according to any one of Statements 106-147, wherein the thermostable mucosal vaccine comprises at least 90% by weight petrolatum carrier.

Statement 155. The composition according to any one of Statements 106-154, wherein the liquid comprising at least one antigen or antigen encoding composition comprises one or more components selected from the group consisting of lactose, sucrose, glycine, monosodium glutamate, human serum albumin, and bovine serum albumin.

Statement 156. The composition according to any one of Statements 106-155, wherein the at least one antigen or antigen encoding composition is selected from the group consisting of a live attenuated vaccine (LAV), an inactivated (killed antigen) vaccine, a subunit (purified antigen) vaccine, a toxoid (inactivated toxin) vaccine, a bacteriophage, a conjugate vaccine composition, a mRNA vaccine composition, a viral vector vaccine composition, an antigen carried or delivered by a bacteriophage, an antigen carried or delivered by a recombinant vesicular stomatitis virus (rVSV) vector, and any combination thereof.

Statement 157. The composition according to any one of Statements 106-156, wherein the at least one antigen or antigen encoding composition is operable, upon administration to a subject, to cause an immune response in a subject sufficient to prevent, mitigate, or treat one or more infections selected from the group consisting of Marburg Virus, Ebola Zaire, Ebola Sudan, Lassa Virus, Buruli Ulcers, Leprosy, SARS-CoV-2 virus, and any combination thereof.

Statement 158. The composition according to any one of Statements 106-157, wherein the liquid comprising at least one antigen or antigen encoding composition is a liquid vaccine effective for the prevention, mitigation, or treatment of Marburg Virus, Ebola Zaire, Ebola Sudan, Lassa Fever, Buruli Ulcers, Leprosy, SARS-CoV-2 virus, and any combination thereof.

Statement 159. The composition according to any one of Statements 106-158, wherein the liquid comprising at least one antigen or antigen encoding composition is a Marburg virus vaccine.

Statement 160. The composition according to any one of Statements 106-158, wherein the liquid comprising at least one antigen or antigen encoding composition is a recombinant vesicular stomatitis virus (rVSV) Ebola vaccine.

Statement 161. The composition according to any one of Statements 106-158, wherein the liquid comprising at least one antigen or antigen encoding composition is a recombinant vesicular stomatitis virus (rVSV) SARS-CoV-2 vaccine.

Statement 162. The composition according to any one of Statements 106-158, wherein the liquid comprising at least one antigen or antigen encoding composition is a Lassa virus vaccine.

Statement 163. The composition according to any one of Statements 106-158, wherein the liquid comprising at least one antigen or antigen encoding composition is a Leprosy vaccine.

Statement 164. The composition according to any one of Statements 106-158, wherein the liquid comprising at least one antigen or antigen encoding composition is a Buruli Ulcer vaccine.

Statement 165. A method of vaccinating a subject, the method comprising: administering the thermostable mucosal vaccine composition according to any one of claims 106-164 to a mucosa or a mucosal membrane of the subject.

Statement 166. A method of causing an immune response in a subject, the method comprising: administering the thermostable mucosal vaccine composition according to any one of claims 106-164 to a mucosa or a mucosal membrane of the subject.

Statement 167. The method according to Statement 165 or Statement 166, wherein administering the thermostable mucosal vaccine composition to the mucosa or a mucosal membrane of the subject comprises oral, nasal, rectal, or vaginal administration.

Statement 168. The method according to Statement 165 or Statement 166, wherein administering the thermostable mucosal vaccine composition to the mucosa or a mucosal membrane of the subject comprises intranasal administration.

Statement 169. A method for determining the effectiveness of a vaccine formulation, the vaccine formulation comprising a recombinant virus in a carrier, the recombinant virus operable to express one or more antigens from a disease-causing agent in a subject in need of vaccination, the method comprising: mixing the vaccine formulation with mineral oil and sterile glass beads to form a first dilution formulation; performing a 10-fold serial dilution of the first dilution formulation using a known concentration of media to form a 10-fold vaccine formulation dilution; plating the 10-fold vaccine formulation dilution onto a multi-well plate prepared with a host cell line monolayer; plating the media and the oil-based carrier onto a multi-well plate prepared with the host cell line monolayer as a control; scoring, microscopically, the wells for viral cytopathic effect, cytotoxic effects, and contamination using the TCID₅₀ Spearman-Karber method to determine the effectiveness of the vaccine formulation.

Statement 170. The method according to Statement 169, wherein the recombinant virus is a vesicular stomatitis virus (VSV) vector.

Statement 171. The method according to Statement 169, wherein the carrier is an oil-based carrier.

Statement 172. The method according to Statement 171, wherein the carrier is petrolatum and/or mineral oil.

Statement 173. The method according to Statement 169, wherein the vaccine formulation comprises the recombinant virus suspended in a petrolatum or mineral oil carrier.

Statement 174. The method according to Statement 169, wherein the known concentration of media is 2% FBS DMEM.

1. A method of preparing a thermostable mucosal vaccine composition, the method comprising:
combining a mucosal vaccine composition with a petrolatum carrier to produce a thermostable mucosal vaccine composition.

2. The method according to claim 1, wherein the vaccine composition is a liquid vaccine composition comprising at least one antigen or antigen-encoding composition, the method comprising:
combining the liquid vaccine composition with the petrolatum carrier to produce a thermostable vaccine composition such that the thermostable vaccine composition comprises at least 80% by weight petrolatum.

3. The method according to claim 2, wherein the vaccine composition is suspended or substantially dispersed in the petrolatum carrier.

4. The method according to claim 3, wherein the vaccine composition is selected from the group consisting of: live attenuated vaccine (LAV) compositions, inactivated vaccine compositions, subunit vaccine compositions, conjugate vaccine compositions, toxoid vaccine compositions, mRNA vaccine compositions, viral vector vaccine compositions.

5. The method according to claim 4, wherein the thermostable vaccine composition is for mucosal use or administration.

6. The method according to claim 5, wherein the thermostable vaccine composition is medically effective when stored at a temperature of about 4 degrees Celsius for 28 days.

7. The method according to claim 5, wherein the thermostable vaccine composition is medically effective after exposed to ambient temperatures for 14 days following removal from temperature-controlled storage.

8. The method according to claim 2, wherein the combining the liquid vaccine composition with a petrolatum carrier comprises:
a) providing a liquid comprising at least one antigen or antigen encoding composition;
b) heating petrolatum or a composition comprising petrolatum to a temperature of from about 37 degrees Celsius to about 40 degrees Celsius to give a melted petrolatum carrier composition;
c) heating the liquid comprising at least one antigen or antigen encoding composition to a temperature equal to or less than the temperature of the melted petrolatum carrier composition to give a heated liquid antigen composition,
d) mixing the melted petrolatum carrier composition and the heated liquid antigen composition to give a melted mixture; and
e) allowing the melted mixture to cool to room temperature to provide a petrolatum mucosal vaccine composition.

9. The method according to claim 2, wherein the combining a liquid vaccine composition with a petrolatum carrier comprises:
a) providing a liquid comprising at least one antigen or antigen encoding composition;
b) heating petrolatum or a composition comprising petrolatum to a temperature of from about 37 degrees Celsius to about 40 degrees Celsius to give a melted petrolatum carrier composition;
c) heating the liquid comprising at least one antigen or antigen encoding composition to a temperature that is about 1 degree C. to about 5 degrees Celsius higher than the temperature of the melted petrolatum carrier composition to give a heated liquid antigen composition,
d) mixing the melted petrolatum carrier composition and the heated liquid antigen composition to give a melted mixture; and
e) allowing the melted mixture to cool to room temperature to provide a petrolatum mucosal vaccine composition.

10. A method of vaccinating a subject, the method comprising:
administering a petrolatum mucosal vaccine to a mucosa or a mucosal membrane of the subject, wherein the petrolatum mucosal vaccine comprises a liquid comprising at least one antigen or antigen encoding composition suspended or substantially dispersed in a petrolatum carrier.

11. The method according to claim 8, wherein administering the petrolatum mucosal vaccine to the mucosa or a mucosal membrane of the subject comprises oral, nasal, rectal, or vaginal administration.

12. The method according to claim 9, further comprising:
combining a liquid comprising at least one antigen or antigen encoding composition with a petrolatum carrier to form a petrolatum mucosal vaccine; and
administering the petrolatum mucosal vaccine to a mucosa or a mucosal membrane of the subject.

13. The method according to claim 11, wherein the at least one antigen or antigen encoding composition is selected from the group consisting of a live attenuated vaccine (LAV), an inactivated (killed antigen) vaccine, a subunit (purified antigen) vaccine, a toxoid (inactivated toxin) vaccine, a bacteriophage, an antigen carried or delivered by a bacteriophage, a conjugate vaccine composition, a mRNA vaccine composition, a viral vector vaccine composition, an antigen carried or delivered by a recombinant vesicular stomatitis virus (rVSV) vector, and any combination thereof.

14. The method according to claim 11, wherein the at least one antigen or antigen encoding composition is operable, upon administration to a subject, to cause an immune response in a subject sufficient to prevent, mitigate, or treat one or more infections selected from the group consisting of Marburg Virus, Ebola Zaire, Ebola Sudan, Lassa Virus, Buruli Ulcers, Leprosy, SARS-CoV-2 virus, and any combination thereof.

15. The method according to claim 11, wherein the liquid comprising at least one antigen or antigen encoding composition is a liquid vaccine effective for the prevention, mitigation, or treatment of Marburg Virus, Ebola Zaire, Ebola Sudan, Lassa Fever, Buruli Ulcers, Leprosy, SARS-CoV-2 virus, and any combination thereof.

16. A thermostable mucosal vaccine composition comprising:
a liquid comprising at least one antigen or antigen encoding composition; and
at least 80% by weight petrolatum carrier.

17. The composition according to claim 16, wherein the liquid comprising at least one antigen or antigen encoding composition is suspended or substantially dispersed in the petrolatum in the form of nanodroplets comprising the liquid and at least one antigen or antigen encoding composition.

18. The composition according to claim 16, wherein the at least one antigen or antigen encoding composition is selected from the group consisting of a live attenuated vaccine (LAV), an inactivated (killed antigen) vaccine, a subunit (purified antigen) vaccine, a toxoid (inactivated toxin) vaccine, a bacteriophage, a conjugate vaccine composition, a mRNA vaccine composition, a viral vector vaccine composition, an antigen carried or delivered by a bacteriophage, an antigen carried or delivered by a recombinant vesicular stomatitis virus (rVSV) vector, and any combination thereof.

19. The composition according to claim 16, wherein the thermostable mucosal vaccine composition is stable and effective when stored at a temperature of from about 2 degrees Celsius to about 8 degrees Celsius for a predetermined period of time prior to administration to a subject, wherein the predetermined period of time is at least 3 months.

20. The composition according to claim 16, wherein the thermostable mucosal vaccine composition is stable and effective when stored at a temperature of from about 8.5 degrees Celsius to about 25 degrees Celsius for a predetermined period of time prior to administration to a subject, wherein the predetermined period of time is greater than 12 hours.
